(19) United States
(12) Patent Application Publication
(10) Pub. No.: US 2021/0214368 A1
(43) Pub. Date: Jul. 15, 2021

(54) COMPOUNDS FOR THIOL-TRIGGERED COS AND/OR H2S RELEASE AND METHODS OF MAKING AND USING THE SAME

(71) Applicant: University of Oregon, Eugene, OR (US)

(72) Inventors: Michael D. Pluth, Eugene, OR (US); Yu Zhao, Springfield, OR (US); Matthew M. Cerda, Berkeley, CA (US)

(73) Assignee: University of Oregon, Eugene, OR (US)

(21) Appl. No.: 15/733,818

(22) PCT Filed: Jan. 30, 2019
(86) PCT No.: PCT/US2019/015833

§ 371 Date: Nov. 30, 2020

Related U.S. Application Data
(60) Provisional application No. 62/679,425, filed on Jun. 1, 2018.

(51) Int. Cl. C07D 493/10; G01N 21/64; G01N 33/00; C07D 285/01

(52) U.S. Cl. CPC C07D 493/10; G01N 21/6486; C09K 11/06; C07D 285/01; G01N 33/0044

(57) ABSTRACT

Disclosed herein are embodiments of a compound that is capable of releasing COS and/or H₂S upon reaction with a thiol-containing compound. The compound embodiments also can produce a detectable signal (e.g., a fluorescent signal) substantially concomitantly with COS and/or H₂S release and/or can release an active agent, such as a therapeutic agent. Methods of making and using the compound embodiments also are disclosed.

### CROSS REFERENCE TO RELATED APPLICATION

This application claims priority to and the benefit of the earlier filing date of U.S. Provisional Application No. 62/679,425, filed on Jun. 1, 2018, the entirety of which is incorporated herein by reference.

### ACKNOWLEDGMENT OF GOVERNMENT SUPPORT

This invention was made with government support under Contract No. R01GM113030 awarded by the National Institutes of Health. The government has certain rights in the invention.

### FIELD

The present disclosure concerns compound embodiments that can release COS, H₂S, a detectable signal, and/or an active agent upon interaction of a thiol compound, as well as methods of making and using such compound embodiments.

### BACKGROUND

Hydrogen sulfide has been recognized as an important biological molecule and plays important biological and pharmacological roles in different conditions associated with human health. For example, H₂S has been implicated in hypertension, diabetes, diseases of mental deficiency, asthma, stroke, and other conditions. For example, administration of H₂S results in reduction in blood pressure in hypertensive mice.

Although convenient, direct administration of H₂S or sulfide-containing salts leads to a large burst of released H₂S, which is quickly metabolized/oxidized by cellular components as part of a toxicological 25 response, and merely results in a disruption of redox homeostasis rather than elevated H₂S levels. There exists a need in the art for an H₂S delivery platform that provides the ability to control the amount and speed of H₂S delivery.

### SUMMARY

Disclosed herein are compound embodiments capable of releasing COS and/or H₂S upon reaction with a thiol-containing compound. In particular disclosed embodiments, the compounds also are capable of releasing a detectable signal and/or an active agent. In some embodiments, the compound has a structure satisfying any one or more of the Formulas disclosed herein.

Also disclosed are embodiments of a pharmaceutical composition comprising a compound and a pharmaceutically acceptable excipient. Also disclosed are embodiments of a method, comprising exposing a sample or a subject to a compound of the present disclosure, or a pharmaceutical composition disclosed herein. In some embodiments, the method can further comprise analyzing the sample or the subject to detect a reaction between the compound and a thiol-containing compound that is inherently present in the subject or the sample, or that is added to the subject or the sample, wherein the reaction produces a detectable signal, COS, H₂S, or a combination thereof. In some embodiments, analyzing comprises detecting and/or measuring a fluorescence change, or a change in concentration of H₂S, or a combination thereof. In some embodiments, the method can further comprise measuring an amount of H₂S released from the compound. The sample can be a biological sample selected from a cell, tissue, and/or bodily fluid.

In some embodiments, the method comprises exposing a subject that has or is at risk of developing a disease associated with H₂S deficiency or H₂S misregulation. The disease can be a cardiovascular disease, diabetes, inflammation, a neurological disease, cancer, a disease involving insufficient wound healing, erectile dysfunction, or any combinations thereof. Cardiovascular diseases can include heart failure, myocardial reperfusion injury, atherosclerosis, hypertension, hypertrophy, or any combinations thereof.

The foregoing and other objects and features of the present disclosure will become more apparent from the following detailed description, which proceeds with reference to the accompanying figures.

### BRIEF DESCRIPTION OF THE DRAWINGS

FIG. 1 is a schematic illustration of a method of using a representative compound embodiment described herein, wherein the compound releases COS (which is then converted to H₂S) and a fluorescent signal upon interaction with a cellular thiol compound.

FIGS. 2A-2D are graphs showing time-dependent fluorescence spectra (FIG. 2A); UV-vis spectra (FIG. 2B) of a representative compound embodiment (10 μM); Cys-dependent (0-200 μM) fluorescence turn on of the representative compound embodiment (10 μM) in PBS (FIG. 2C), and methylene blue (MB) measurement of H₂S release from the representative compound embodiment (10 μM) upon Cys (100 μM) activation (FIG. 2D).

FIGS. 3A and 3B are graphs showing time-dependent fluorescence turn-on of a compound embodiment (FIG. 3A) and the correlation between fluorescence measurement and methylene blue detection (FIG. 3B).

FIG. 4 is a graph of fluorescence turn on of certain compound embodiments described herein.

FIG. 5 is a graph of fluorescence turn on of a representative compound embodiment disclosed herein in the presence of cellular reactive sulfur, oxygen, and nitrogen species (RSONs).

FIG. 6 is a graph showing GSH-dependent fluorescence turn on of compound 202a (10 μM) in PBS.

FIG. 7 includes images showing H₂S delivery from a representative compound embodiment in HeLa cells.

FIG. 8 is a graph showing results from evaluating the cytotoxicity of compound 202a in HeLa cells.

FIG. 9 is a graph showing results from evaluating the cytotoxicity of compound 202a, control compound 204, and BnSH in RAW 264.7 cells.

FIG. 10 is a graph showing cytoprotective activity of a representative compound embodiment against LPS-induced inflammation.

FIG. 11 is a graph showing the effects of control compound 204 and H₂S releasing by-products on LPS-induced NO₂— accumulation.

FIG. 12 illustrates a collection of HPLC traces providing a reaction analysis of 10 μM compound 202a (top trace); 100 μM BnSH containing Bn₂S₂ due to aerobic oxidation (second trace from top); 20 μM Bn₂S₂ (third trace from top); 10 μM fluorescein (fourth trace from top); 0.1% (v/v) DMSO in 10 mM PBS (pH 7.4) (second trace from bottom); and a reaction aliquot after 1 hour (bottom trace).

FIG. 13 is a graph showing GSH-dependent H₂S release from compound 306a.

FIG. 14 is a graph showing thiol-triggered H₂S release using various thiols in combination with compound 306a.

FIG. 15 is a graph showing H₂S release from compound 306a in the presence of cellular reactive species.

FIG. 16 is a graph showing GSH-triggered H₂S release from compound 900.

FIG. 17 is a graph showing that compound 404c reacts with thiols to release two equivalents of COS, which is converted to H₂S by the ubiquitous enzyme carbonic anhydrase (CA).

FIG. 18 is a graph of H₂S release from various compound embodiments disclosed herein (compounds 306a-306e).

FIG. 19 is a graph showing Cys-triggered H₂S release from a series of compound embodiments disclosed herein (compounds 404a, 404f-404i, and 404l).

FIG. 20 are images showing H₂S delivery from compound embodiments (namely, compound 306a) of the present disclosure in HeLa cells, wherein the HeLa cells were treated with Hoechst dye and SF7-AM (5 μM) in DMEM only for 5 minutes and then with DMEM only for 30 minutes (top row) or DMEM containing the donor (50 μM) for 30 minutes (bottom row), after which cells were washed with PBS and cell images were taken in PBS using a fluorescent microscope.

FIG. 21 is a graph showing H₂S release from compound 900, which comprises a therapeutic agent.

### DETAILED DESCRIPTION

### I. Overview of Terms

The following explanations of terms are provided to better describe the present disclosure and to guide those of ordinary skill in the art in the practice of the present disclosure. As used herein, “comprising” means “including” and the singular forms “a” or“an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

To facilitate review of the various embodiments of the disclosure, the following explanations of specific terms are provided. Certain functional group terms include a symbol“−” which is used to show how the defined functional group attaches to, or within, the compound to which it is bound. Also, a dashed bond (i.e., “ - - - ”) as used in certain formulas described herein indicates an optional bond (that is, a bond that may or may not be present). A person of ordinary skill in the art would recognize that the definitions provided below and the compounds and formulas included herein are not intended to include impermissible substitution patterns (e.g., methyl substituted with 5 different groups, and the like). Such impermissible substitution patterns are easily recognized by a person of ordinary skill in the art. In formulas and compounds disclosed herein, a hydrogen atom is present and completes any formal valency requirements (but may not necessarily be illustrated) wherever a functional group or other atom is not illustrated. For example, a phenyl ring that is drawn as

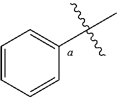 [SMILES: *c1ccccc1]

comprises a hydrogen atom attached to each carbon atom of the phenyl ring other than the “a” carbon, even though such hydrogen atoms are not illustrated. Any functional group disclosed herein and/or defined above can be substituted or unsubstituted, unless otherwise indicated herein.

Acyl Halide: —C(O)X, wherein X is a halogen, such as Br, F, I, or Cl.

Aldehyde: —C(O)H.

Aliphatic: A hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well.

Aliphatic-aromatic: An aromatic group that is or can be coupled to a compound disclosed herein, wherein the aromatic group is or becomes coupled through an aliphatic group.

Aliphatic-aryl: An aryl group that is or can be coupled to a compound disclosed herein, wherein the aryl group is or becomes coupled through an aliphatic group.

Aliphatic-heteroaryl: A heteroaryl group that is or can be coupled to a compound disclosed herein, wherein the heteroaryl group is or becomes coupled through an aliphatic group.

Alkenyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z).

Alkoxy: —O-aliphatic, such as —O-alkyl, —O-alkenyl, —O-alkynyl; with exemplary embodiments including, but not limited to, methoxy, ethoxy, n-propoxy, isopropoxy, n-butoxy, t-butoxy, sec-butoxy, n-pentoxy (wherein any of the aliphatic components of such groups can comprise no double or triple bonds, or can comprise one or more double and/or triple bonds).

Alkyl: A saturated monovalent hydrocarbon having at least one carbon atom to 50 carbon atoms (C₁₋₅₀), such as one to 25 carbon atoms (C₁₋₂₅), or one to ten carbon atoms (C₁₋₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl).

Alkynyl: An unsaturated monovalent hydrocarbon having at least two carbon atom to 50 carbon atoms (C₂₋₅₀), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkyny).

Amide: —C(O)NR^{a}R^{b} or —NR^{a}C(O)R^{b} wherein each of R^{a} and R^{b} independently is selected from hydrogen, aliphatic, heteroaliphatic, haloaliphatic, haloheteroaliphatic, aromatic, or an organic functional group.

Amino: —NR^{a}R^{b}, wherein each of R^{a} and R^{b} independently is selected from hydrogen, aliphatic, heteroaliphatic, haloaliphatic, haloheteroaliphatic, aromatic, or an organic functional group.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridiny); that is, at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

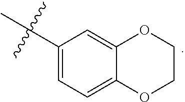 [SMILES: *c1ccc2c(c1)OCCO2 ; "."]

However, in certain examples, context or express disclosure may indicate that the point of attachment is through a non-aromatic portion of the condensed ring system. For example,

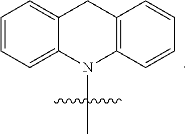 [SMILES: *N1c2ccccc2Cc2ccccc21 ; "."]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Aromatic groups may be substituted with one or more groups other than hydrogen, such as aliphatic, heteroaliphatic, haloaliphatic, haloheteroaliphatic, aromatic, or an organic functional group.

Aryl: An aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, heteroaliphatic, haloaliphatic, haloheteroaliphatic, aromatic, or an organic functional group.

Aroxy: —O-aromatic.

Azo: —N═NR^{a} wherein R^{b} is hydrogen, aliphatic, heteroaliphatic, haloaliphatic, haloheteroaliphatic, aromatic, or an organic functional group.

Carbamate: —OC(O)NR^{a}R^{b}, wherein each of R^{a} and R^{b} independently is selected from hydrogen, aliphatic, heteroaliphatic, haloaliphatic, haloheteroaliphatic, aromatic, or an organic functional group.

Carboxyl: —C(O)OH.

Carboxylate: —C(O)— or salts thereof, wherein the negative charge of the carboxylate group may be balanced with an M⁺ counterion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Cyano: —CN.

Detectable Moiety: A component of a compound embodiment that provides a detectable signal. In some embodiments, the detectable moiety can provide the detectable signal when attached to a compound embodiment. In some embodiments, the detectable moiety can provide the detectable signal when cleaved from a compound embodiment.

Detectable Signal: A signal (e.g., a color change, an increase or decrease in fluorescence, an increase or decrease in phosphorescence or other type of luminescence, and the like) that occurs (or is quenched) when a compound disclosed herein comprising a detectable moiety (e.g., a fluorophore or a dye) reacts with a reactive compound. In some embodiments, a detectable signal is visible to the naked eye or is visible using an analytical detection technique.

Disulfide: —SSR^{a}, wherein R^{a} is selected from hydrogen, aliphatic, heteroaliphatic, haloaliphatic, haloheteroaliphatic, aromatic, or an organic functional group.

Dithiocarboxylic: —C(S)SR^{a} wherein R^{b} is selected from hydrogen, aliphatic, heteroaliphatic, haloaliphatic, haloheteroaliphatic, aromatic, or an organic functional group.

Ester: —C(O)OR^{a} or —OC(O)R^{a}, wherein R^{a} is selected from aliphatic, heteroaliphatic, haloaliphatic, haloheteroaliphatic, aromatic, or an organic functional group.

Ether: -aliphatic-O-aliphatic, -aliphatic-O-aromatic, -aromatic-O-aliphatic, or -aromatic-O-aromatic.

Fluorophore: A functional group or portion of a compound that causes the compound (or a sample or composition comprising the compound), to fluoresce. In some embodiments, the fluorophore can fluoresce when the compound (or a sample or composition comprising the compound) is exposed to an excitation source or after being cleaved from a compound embodiment. Representative fluorophores can include, but are not limited to, a xanthene derivative (e.g., fluorescein, rhodamine, eosin, Texas red, Oregon green, or the like), cyanine or a cyanine derivative (e.g., indocarbocyanine, oxacarbocyanine, thiacarbocyanine, merocyanine, Cy3, or Cy5), a naphthalene derivative (e.g., dansyl, prodan, and the like), coumarin and derivatives thereof (e.g., hydroxycoumarin, aminocoumarin, methoxycoumarin, and the like), oxadiazole derivatives (e.g., pyridyloxazole, nitrobenzoxadiazole, benzoxadiazole, and the like), anthracene derivatives, pyrene derivatives (e.g., cascade blue), oxazine derivatives (e.g., Nile red, Nile blue, cresyl violet, and the like), acridine derivatives (e.g., auramine, crystal violet, malachite green, and the like), fluorone dyes (e.g., rhodamine, rhodol, methylrhodol), isoquinoline dyes (e.g., 2-(2-methoxyethyl)-1H-benzo[de]isoquinoline-1,3(2H)-dione), a naphthalimide compound (e.g., naphthalimide or 4-(2-methoxyethoxy)-N-butyl-1,8-naphthalimide), a chromenone dye (e.g., 4-methyl-2H-chromen-2-one), and tetrapyrrole derivatives (e.g., porphin, phthalocyanine, and the like) and in some embodiments can be methylrhodol, 2-(2-methoxyethyl)-1H-benzo[de]isoquinoline-1,3(2H)-dione, 4-methyl-2H-chromen-2-one, coumarin, naphthalimide, fluorescein, rhodamine, rhodol, Cy3, or Cy5. In some embodiments, compound embodiments of the present disclosure comprise a precursor to such fluorophore groups. Also, fluorophore compound embodiments can be described as heteroaryl and/or heteroaliphatic (e.g., heterocyclic) groups in the present disclosure.

Halo (or halide or halogen): Fluoro, chloro, bromo, or iodo.

Haloaliphatic: An aliphatic group wherein one or more hydrogen atoms, such as one to 10 hydrogen atoms, independently is replaced with a halogen atom, such as fluoro, bromo, chloro, or iodo.

Haloaliphatic-aryl: An aryl group that is or can be coupled to a compound disclosed herein, wherein the aryl group is or becomes coupled through a haloaliphatic group.

Haloaliphatic-heteroaryl: A heteroaryl group that is or can be coupled to a compound disclosed herein, wherein the heteroaryl group is or becomes coupled through a haloaliphatic group.

Haloalkyl: An alkyl group wherein one or more hydrogen atoms, such as one to 10 hydrogen atoms, independently is replaced with a halogen atom, such as fluoro, bromo, chloro, or iodo. In an independent embodiment, haloalkyl can be a CX₃ group, wherein each X independently can be selected from fluoro, bromo, chloro, or iodo.

Heteroaliphatic: An aliphatic group comprising at least one heteroatom to 20 heteroatoms, such as one to 15 heteroatoms, or one to 5 heteratoms, which can be selected from, but not limited to oxygen, nitrogen, sulfur, silicon, boron, selenium, phosphorous, and oxidized forms thereof within the group. Alkoxy, ether, amino, disulfide, peroxy, and thioether groups are exemplary (but non-limiting) examples of heteroaliphatic. In some embodiments, a fluorophore can also be described herein as a heteraliphatic group, such as when the heteroaliphatic group is a heterocyclic group.

Heteroaliphatic-aryl: An aryl group that is or can be coupled to a compound disclosed herein, wherein the aryl group is or becomes coupled through a heteroaliphatic group.

Heteroaryl: An aryl group comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, haloaliphatic, haloheteroaliphatic, aromatic, or an organic functional group. In some embodiments, a fluorophore can also be described herein as a heteroaryl group.

Heteroatom: An atom other than carbon or hydrogen, such as (but not limited to) oxygen, nitrogen, sulfur, silicon, boron, selenium, or phosphorous. In particular disclosed embodiments, such as when valency constraints do not permit, a heteroatom does not include a halogen atom.

Ketone: —C(O)R^{a}, wherein R^{a} is selected from aliphatic, heteroaliphatic, haloaliphatic, haloheteroaliphatic, aromatic, or an organic functional group.

Organic Functional Group: A functional group that may be provided by any combination of aliphatic, heteroaliphatic, aromatic, haloaliphatic, and/or haloheteroaliphatic groups, or that may be selected from, but not limited to, aldehyde; aroxy; acyl halide; halogen; nitro; cyano; azide; carboxyl (or carboxylate); amide; ketone; carbonate; imine; azo; carbamate; hydroxyl; thiol; sulfonyl (or sulfonate); oxime; ester; thiocyanate; thioketone; thiocarboxylic acid; thioester dithiocarboxylic acid or ester; phosphonate; phosphate; silyl ether; sulfinyl; thial; or combinations thereof.

Oxime: —CR^{a}═NOH, wherein R^{a} is hydrogen, aliphatic, heteroaliphatic, haloaliphatic, haloheteroaliphatic, aromatic, or an organic functional group.

Peroxy: —O—OR^{a} wherein R^{a} is hydrogen, aliphatic, heteroaliphatic, haloaliphatic, haloheteroaliphatic, aromatic, or an organic functional group.

Pharmaceutically Acceptable Excipient: A substance, other than a compound that is included in a formulation of the compound. As used herein, an excipient may be incorporated within particles of a pharmaceutical composition, or it may be physically mixed with particles of a pharmaceutical composition. An excipient also can be in the form of a solution, suspension, emulsion, or the like. An excipient can be used, for example, to dilute an active agent and/or to modify properties of a pharmaceutical composition. Excipients can include, but are not limited to, antiadherents, binders, coatings, enteric coatings, disintegrants, flavorings, sweeteners, colorants, lubricants, glidants, sorbents, preservatives, adjuvants, carriers or vehicles. Excipients may be starches and modified starches, cellulose and cellulose derivatives, saccharides and their derivatives such as disaccharides, polysaccharides and sugar alcohols, protein, synthetic polymers, crosslinked polymers, antioxidants, amino acids or preservatives. Exemplary excipients include, but are not limited to, magnesium stearate, stearic acid, vegetable stearin, sucrose, lactose, starches, hydroxypropyl cellulose, hydroxypropyl methylcellulose, xylitol, sorbitol, maltitol, gelatin, polyvinylpyrrolidone (PVP), polyethylene glycol (PEG), tocopheryl polyethylene glycol 1000 succinate (also known as vitamin E TPGS, or TPGS), carboxy methyl cellulose, dipalmitoyl phosphatidyl choline (DPPC), vitamin A, vitamin E, vitamin C, retinyl palmitate, selenium, cysteine, methionine, citric acid, sodium citrate, methyl paraben, propyl paraben, sugar, silica, talc, magnesium carbonate, sodium starch glycolate, tartrazine, aspartame, benzalkonium chloride, sesame oil, propyl gallate, sodium metabisulphite or lanolin. In independent embodiments, water is not intended as a pharmaceutically acceptable excipient.

Phosphate: —O—P(O)(OR^{a})₂, wherein each R^{a} independently is hydrogen, aliphatic, heteroaliphatic, haloaliphatic, haloheteroaliphatic, aromatic, or an organic functional group; or wherein one or moreR^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Phosphonate: —P(O)(OR^{a})₂, wherein each R^{a} independently is hydrogen, aliphatic, heteroaliphatic, haloaliphatic, haloheteroaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²]_{0.5}.

Reactive Compound: A compound that reacts (typically via nucleophilic attack) with a compound embodiment so as to initiate COS and/or H₂S release from the compound embodiment. In some embodiments, the reactive compound comprises a thiol group and can be a thiol-containing compound inherently present in a subject or sample, or it can be a thiol compound provided by an external source.

Silyl Ether —OSiR^{a}R^{b}, wherein each of R^{a} and R^{b} independently is selected from hydrogen, aliphatic, heteroaliphatic, haloaliphatic, haloheteroaliphatic, aromatic, or an organic functional group.

Subject: Mammals and other animals, such as humans, companion animals (e.g., dogs, cats, rabbits, etc), utility animals, and feed animals; thus, disclosed methods are applicable to both human therapy and veterinary applications.

Sulfinyl: —S(O)R^{a}, wherein R^{a} is selected from hydrogen, aliphatic, heteroaliphatic, haloaliphatic, haloheteroaliphatic, aromatic, or an organic functional group.

Sulfonyl: —SO₂R^{a}, wherein R^{a} is selected from hydrogen, aliphatic, heteroaliphatic, haloaliphatic, haloheteroaliphatic, aromatic, or an organic functional group.

Sulfonamide: —SO₂NR^{a}R^{b} or —N(R^{a})SO₂R^{b}, wherein each of R^{a} and R^{b} independently is selected from hydrogen, aliphatic, heteroaliphatic, haloaliphatic, haloheteroaliphatic, aromatic, or an organic functional group.

Sulfonate: —SO₃—, wherein the negative charge of the sulfonate group may be balanced with an M⁺ counter ion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, haloheteroaliphatic, or aromatic; or an alkaline earth ion, such as [Ca²⁺]_{0.5}, [Mg²⁺]_{0.5}, or [Ba²⁺]_{0.5}.

Thial: —C(S)H.

Thiocarboxylic acid: —C(O)SH, or —C(S)OH.

Thiocyanate: —S—CN or —N═C═S.

Thioester: —C(O)SR^{a} or —C(S)OR^{a} wherein R^{a} is selected from hydrogen, aliphatic, heteroaliphatic, haloaliphatic, haloheteroaliphatic, aromatic, or an organic functional group.

Thioether —S-aliphatic or —S-aromatic, such as —S-alkyl, —S-alkenyl, —S-alkynyl, —S-aryl, or —S— heteroaryl; or -aliphatic-S-aliphatic, -aliphatic-S-aromatic, -aromatic-S-aliphatic, or -aromatic-S-aromatic.

Thioketone: —C(S)R^{a} wherein R^{a} is selected from hydrogen, aliphatic, heteroaliphatic, haloaliphatic, haloheteroaliphatic, aromatic, or an organic functional group.

Treating/Treatment: Treatment of a disease or condition of interest in a subject, particularly a human or canine having the disease or condition of interest, and includes by way of example, and without limitation:
(i) prophylactic administration to prevent the disease or condition from occurring in a subject, or to ameliorate symptoms associated with the condition if required in particular, when such subject is predisposed to the condition but has not yet been diagnosed as having it;
(ii) inhibiting the disease or condition, for example, arresting or slowing its development;
(iii) relieving the disease or condition, for example, causing regression of the disease or condition or a symptom thereof; or
(iv) stabilizing the disease or condition.

As used herein, the terms “disease” and “condition” can be used interchangeably or can be different in that the particular malady or condition may not have a known causative agent (so that etiology has not yet been determined) and it is therefore not yet recognized as a disease but only as an undesirable condition or syndrome, where a more or less specific set of symptoms have been identified by clinicians.

### II. Introduction

Real-time tracking of donor activation and H₂S delivery in living systems remains as a key challenge in many technologies due to the inherent limitations of current H₂S detection methods. For example, the colorimetric methylene blue (MB) assay has been widely used to measure H₂S levels, but requires strongly acidic condition, which may trigger H₂S release from acid labile sulfide pools. Similarly, H₂S-selective electrodes are most often used in bulk measurements rather than non-homogenized biological samples. H₂S fluorescent probes have attracted attention in the art due to their high sensitivities and have been used to sense and visualize H₂S in biological samples; however, to date, H₂S fluorescent probes are prone to react with reactive cellular species, such as Cys or glutathione (GSH), which results in either probe consumption or false positive signals. In addition, methods currently used in the art typically consume the H₂S being measured. To conduct live-cell and tissue experiments, there is a need in the art for H₂S donors that deliver H₂S with a concomitant fluorescence response to enable tracking of H₂S delivery by common microscopy techniques.

Aligned with these needs, disclosed herein are new COS/H₂S-releasing compounds that comprise a caged sulfenyl thiocarbonate skeleton

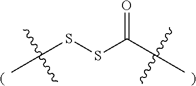

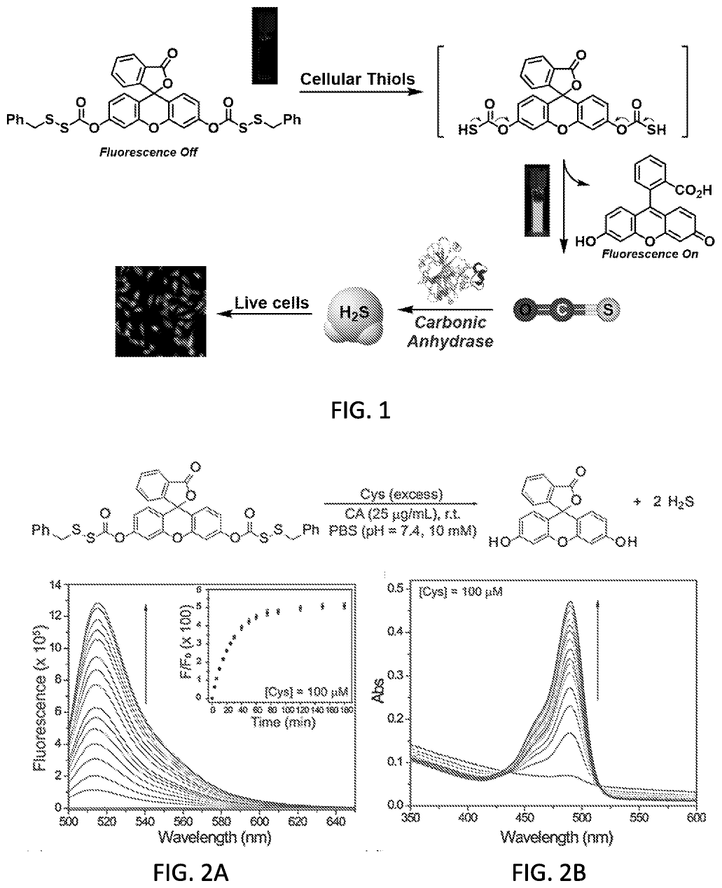

and can serve as fluorescent turn-on and/or active agent releasing compounds. Reactive compounds, such as cellular thiols (e.g., Cys and GSH), can activate the compounds through thiol-mediated disulfide reduction to release COS, which is quickly converted to H₂S by carbonic anhydrase (CA) (FIG. 1). This reduction strategy provides a new activation pathway that has not been used to trigger COS/H₂S release from compound platforms. The compound embodiments of the present disclosure do not generate reactive electrophile by-products upon activation, which provides a significant advance in the field. In addition, certain compound embodiments can provide a concomitant fluorescence turn-on upon compound activation, thus allowing for real-time monitoring and quantification of H₂S release using fluorescence spectroscopy. Additional compound embodiments of the present disclosure can be used to release H₂S as well as an active compound.

### III. Compound Embodiments

Disclosed herein are compound embodiments that are capable of donating COS and/or H₂S upon reaction with a reactive compound, such as a thiol-containing compound. Further, the compounds are capable of releasing an active agent, such as a drug compound; and/or releasing a detectable moiety that exhibits a detectable signal. In particular disclosed embodiments, the compound embodiments of this disclosure have a structure satisfying Formula I, below.

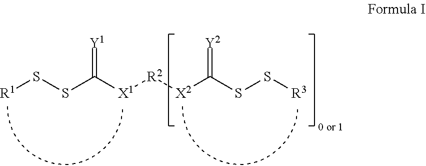

With reference to Formula I, each of Y¹ and Y² independently can be oxygen or sulfur. R¹ and R³ independently can be aliphatic, heteroaliphatic, haloaliphatic, haloheteroaliphatic, aromatic, an active agent, an organic functional group, or any combination thereof; or R¹ and X¹ (and/or R³ and X²), together, can provide a ring (as indicated by the curved dashed bonds in Formula I). In embodiments where R¹ and X¹, together, provide a five-membered ring, R¹ can be C(O), wherein the carbon atom of the carbonyl group is bound to X¹ via a single bond, or R¹ can be CR⁴, wherein the carbon atom of the CR⁴ group is bound to X¹ via a double bond and R⁴ is aliphatic, heteroaliphatic, haloaliphatic, haloheteroaliphatic, aromatic, an active agent, an organic functional group, or any combination thereof. Similarly, when R³ and X², together, provide a five-membered ring, R³ can be C(O), wherein the carbon atom of the carbonyl group is bound to X² via a single bond, or R³ can be CR⁴, wherein the carbon atom of the CR⁴ group is bound to X² via a double bond and R⁴ is aliphatic, heteroaliphatic, haloaliphatic, haloheteroaliphatic, aromatic, an active agent, an organic functional group, or any combination thereof. X¹ and X² independently can be oxygen, nitrogen, or NR (wherein R⁵ is hydrogen, aliphatic, heteroaliphatic, haloaliphatic, haloheteroaliphatic, aromatic, an organic functional group, or a combination thereof) and in some embodiments where X¹ and/or X² are oxygen, R¹ and X¹ (or R³ and X²) do not, together, provide a ring and instead X¹ is bound to R² (and/or X² is bound to R²). In embodiments where X¹ and/or X² are nitrogen, R² may or may not be present depending on the available valency of the nitrogen atom. For example, if R¹ is CR⁴, then R² typically is not present as the nitrogen atom is bound to the CR⁴ group via a double bond. In embodiments wherein R¹ is C(O), then R² can be present. R², if present (such as when X¹ and/or X² is oxygen, or when X¹ is nitrogen and R¹ is C(O) and/or X² is nitrogen and R³ is C(O)), can be a detectable moiety and/or can be selected from hydrogen, aliphatic, heteroaliphatic, haloaliphatic, haloheteroaliphatic, aromatic, an organic functional group, or a combination thereof. In some embodiments, both Y¹ and Y² can be oxygen or both Y¹ and Y² can be sulfur; or one of Y¹ and Y² can be oxygen and the other can be sulfur.

In particular disclosed embodiments, R¹ (and/or R³) is alkyl, heteroaryl, or aryl; or R¹ (and/or R³) can be C(O) or CR⁴ wherein the carbon atom of these groups joins with X¹ (or X²) to provide a five-membered ring. In some embodiments, R² can be a heteroaryl or heterocyclic group, such as a fluorophore (e.g., xanthene derivative; cyanine or a cyanine derivative; a naphthalene derivative; coumarin and derivatives thereof; oxadiazole derivatives; anthracene derivatives, pyrene derivatives; oxazine derivatives; acridine derivatives; fluorone dyes; isoquinoline dyes; a naphthalimide compound; a chromenone dye; or tetrapyrrole derivatives) or a precursor thereof. In some representative embodiments, R² is methylrhodol, 2-(2-methoxyethyl)-1H-benzo[de]isoquinoline-1,3(2H)-dione, 4-methyl-2H-chromen-2-one, coumarin, naphthalimide, fluorescein, rhodamine, rhodol, Cy3, or Cy5. Any of these fluorophore compounds can be bound to X¹ and/or X² via functional groups of the fluorophore.

In some embodiments, the compound can have a structure satisfying any one or more of Formulas III-V, which are discussed below.

In some embodiments, the compound can have a structure satisfying Formula II, with some embodiments having structures satisfying Formula IIA or IIB.

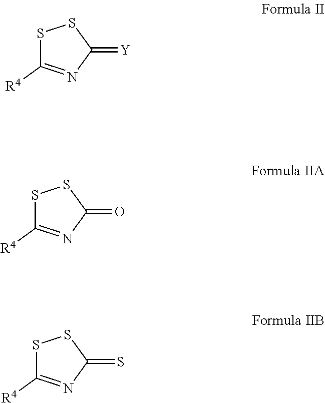

With reference to Formula II, Y is oxygen or sulfur. With reference to Formulas I, IIA, and IIB, R⁴ is aliphatic, heteroaliphatic, haloaliphatic, haloheteroaliphatic, aromatic, an organic functional group, or any combination thereof. In particular embodiments of Formula II, R⁴ is heteroaryl or aryl. In yet additional embodiments of Formula II, R⁴ is -heteroaryl-(Z)ₙ or -aryl-(Z)ₙ, wherein each Z is a substituent other than hydrogen and can be positioned at any position on the heteroaryl and/or aryl ring and n is an integer ranging from 0 to an integer value equal to the number of positions on the heteroaryl group or the aryl group that can be substituted. In embodiments where R⁴ is -heteroaryl-(Z)ₙ or -aryl-(Z)ₙ, and the heteroaryl or the aryl ring are 6-membered rings, Z can be in the ortho, meta, or para position relative to the bond between the R⁴ group and the remainder of Formula II. In some embodiments, n is an integer ranging from 0 to 20, such as 0 to 15, or 0 to, or to 2, or to 15, or 1 to 10 (e.g., 0, 1, 2, 3, 4, 5, 6, 7, 8, 9, 10, 11, 12, 13, 14, 15, 16, 17, 18, 19, 20). Each Z independently can be aliphatic; aromatic; heteroaliphatic (e.g., peroxy; disulfide; alkoxy; ether; thioether amino, such as —NR^{a}R^{b}, wherein R^{a} is aliphatic, heteraliphatic, haloaliphatic, haloheteroaliphatic, aromatic, or an organic functional group and R^{b} is hydrogen, aliphatic, heteroaliphatic, haloaliphatic, haloheteroaliphatic, aromatic, or an organic functional group; or —NR^{a}R^{b}, wherein R^{a} and R^{b} are hydrogen); haloaliphatic; haloheteroaliphatic; an active agent (e.g., naproxen); or an organic functional group, such as, aroxy; aldehyde; acyl halide; halogen; nitro; cyano; azide; carboxyl (or carboxylate); amide; ketone; carbonate; imine; azo; carbamate; hydroxyl; thiol; sulfonyl (or sulfonate); oxime; ester; thiocyanate; thioketone; thiocarboxylic acid; thioester dithiocarboxylic acid or ester phosphonate; phosphate; silyl ether; sulfinyl; thial; or combinations thereof.

In some embodiments, the compound can have a structure satisfying Formula III, with some embodiments having structures satisfying Formula IIIA or IIIB.

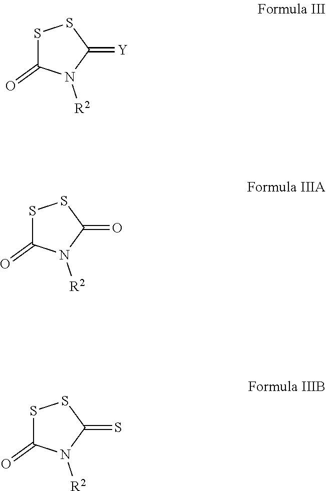

With reference to Formula II, Y can by oxygen or sulfur. With reference to Formulas III, IIIA, and IIIB, R² is hydrogen, aliphatic, heteroaliphatic, haloaliphatic, haloheteroaliphatic, aromatic, an active agent, an organic functional group, or any combination thereof. In some embodiments of Formula III, R² is hydrogen, alkyl, alkenyl, alkynyl, heteroalkyl, heteroalkenyl, heteroalkynyl, heteroaryl, aryl, or combinations thereof. In yet some additional embodiments of Formula III, R² is a detectable moiety (e.g., a fluorophore or precursor thereof), an active agent, -heteroaryl-(Z)ₙ, or -aryl-(Z)ₙ, wherein each Z is a substituent other than hydrogen and n is an integer ranging from 0 to an integer value equal to the number of positions on the heteroaryl group or the aryl group that can be substituted. In embodiments where R² is -heteroaryl-(Z)ₙ or -aryl-(Z)ₙ, and the heteroaryl and aryl ring are 6-membered rings, Z can be in the ortho, meta, or para position relative to the bond between the R² group and nitrogen atom depicted in Formula II. In some embodiments, n is an integer ranging from 0 to 20, such as 0 to 15, or 0 to 10, or 1 to 20, or 1 to 15, or 1 to 10 (e.g., 0, 1, 2, 3, 4, 5, 6, 7, 8, 9, 10, 11, 12, 13, 14, 15, 16, 17, 18, 19, 20). Each Z independently can be aliphatic; aromatic; heteroaliphatic (e.g., peroxy; disulfide; alkoxy; ether thioether; amino, such as —NR^{a}R^{b}, wherein R^{a} is aliphatic, heteroaliphatic, haloaliphatic, haloheteroaliphatic, aromatic, or an organic functional group and R^{b} is hydrogen, aliphatic, heteroaliphatic, haloaliphatic, haloheteroaliphatic, aromatic, or an organic functional group; or —NR^{a}R^{b}, wherein R^{a} and R^{b} are hydrogen); haloaliphatic; haloheteroaliphatic; or an organic functional group, such as, aroxy; aldehyde; acyl halide; halogen; nitro; cyano; azide; carboxyl (or carboxylate); amide; ketone; carbonate; imine; azo; carbamate; hydroxyl; thiol; sulfonyl (or sulfonate); oxime; ester; thiocyanate; thioketone; thiocarboxylic acid; thioester; dithiocarboxylic acid or ester; phosphonate; phosphate; silyl ether; sulfinyl; thial; or combinations thereof. In embodiments where R² is a fluorophore (or precursor thereof), the fluorophore can be a xanthene derivative; cyanine or a cyanine derivative; a naphthalene derivative; coumarin or a derivative thereof; an oxadiazole derivative; an anthracene derivative; a pyrene derivative; an oxazine derivative; an acridine derivative; a fluorone dye; an isoquinoline dye; a naphthalimide compound; a chromenone dye; or a tetrapyrrole derivative (or a precursor thereof of any such compounds). In some representative embodiments, R² is methylrhodol, 2-(2-methoxyethyl)-1H-benzo[de]isoquinoline-1,3(2H)-dione, 4-methyl-2H-chromen-2-one, coumarin, naphthalimide, fluorescein, rhodamine, rhodol, Cy3, or Cy5 or a precursor thereof.

In some embodiments, the compound can have a structure satisfying Formula IV, with some embodiments having structures satisfying Formula IVA or IVB.

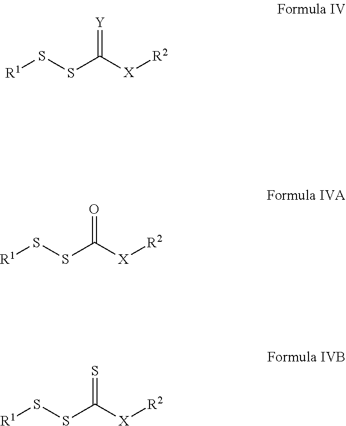

With reference to Formula IV, Y is oxygen or sulfur. With reference to Formulas IV, IVA, and IVB, R¹ is aliphatic, heteroaliphatic, haloaliphatic, haloheteroaliphatic, aromatic, an active agent, an organic functional group, or any combination thereof; R² is hydrogen, aliphatic, heteroaliphatic, haloaliphatic, haloheteroaliphatic, aromatic, or an organic functional group; and X is oxygen or NR⁵ (wherein R⁵ is hydrogen, aliphatic, heteroaliphatic, haloaliphatic, haloheteroaliphatic, aromatic, an organic functional group, or a combination thereof). In some embodiments of Formula IV, R¹ is alkyl, alkenyl, alkynyl, heteroalkyl, heteroalkenyl, heteroalkynyl, heteroaryl, aryl, benzyl, or an active agent, such as

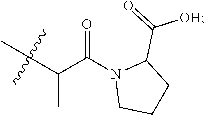 [SMILES: *C(C)C(=O)N1CCCC1C(=O)O]

R² is hydrogen, alkyl, alkenyl, alkynyl, heteroalkyl, heteroalkenyl, heteroalkynyl, heteroaryl, aryl, or combinations thereof. In yet some additional embodiments of Formula IV, each of R¹ and R² independently can be -heteroaryl-(Z)ₙ, or -aryl-(Z)ₙ, wherein each Z is a substituent other than hydrogen and n is an integer ranging from 0 to an integer value equal to the number of positions on the heteroaryl group or the aryl group that can be substituted; or R² can be a detectable moiety (e.g., a fluorophore or precursor thereof). In embodiments where R¹ or R² is -heteroaryl-(Z) or -aryl-(Z)ₙ, and the heteroaryl and aryl ring are 6-membered rings, Z can be in the ortho, meta, or para position relative to the bond between the R² group and X variable atom (or the bond between the R¹ group and the sulfur atom) depicted in Formula I. In some embodiments, n is an integer ranging from 0 to 20, such as 0 to 15, or 0 to 10, or 1 to 20, or 1 to 15, or 1 to 10 (e.g., 0, 1, 2, 3, 4, 5, 6, 7, 8, 9, 10, 11, 12, 13, 14, 15, 16, 17, 18, 19, 20). Each Z independently can be aliphatic; aromatic; heteroaliphatic (e.g., peroxy; disulfide; alkoxy; ether thioether amino, such as —NR^{a}R^{b}, wherein R^{a} is aliphatic, heteroaliphatic, haloaliphatic, haloheteroaliphatic, aromatic, or an organic functional group and R is hydrogen, aliphatic, heteroaliphatic, haloaliphatic, haloheteroaliphatic, aromatic, or an organic functional group; or —NR^{a}R^{b}, wherein R^{a} and R^{b} are hydrogen); haloaliphatic; haloheteroaliphatic; or an organic functional group, such as, aroxy; aldehyde; acyl halide; halogen; nitro; cyano; azide; carboxyl (or carboxylate); amide; ketone; carbonate; imine; azo; carbamate; hydroxyl; thiol; sulfonyl (or sulfonate); oxime; ester; thiocyanate; thioketone; thiocarboxylic acid; thioester; dithiocarboxylic acid or ester; phosphonate; phosphate; silyl ether sulfinyl; thial; or combinations thereof. In embodiments where R² is a fluorophore (or precursor thereof), the fluorophore can be a xanthene derivative; cyanine or a cyanine derivative; a naphthalene derivative; coumarin or a derivative thereof; an oxadiazole derivative; an anthracene derivative; a pyrene derivative; an oxazine derivative; an acridine derivative; a fluorone dye; an isoquinoline dye; a naphthalimide compound; a chromenone dye; or a tetrapyrrole derivative (or a precursor of any such compounds). In some representative embodiments, R² is methylrhodol, 2-(2-methoxyethy)-1H-benzo[de]isoquinoline-1,3(2H)-dione, 4-methyl-2H-chromen-2-one, coumarin, naphthalimide, fluorescein, rhodamine, rhodol, Cy3, or Cy5 or a precursor thereof. In some embodiments, R² is

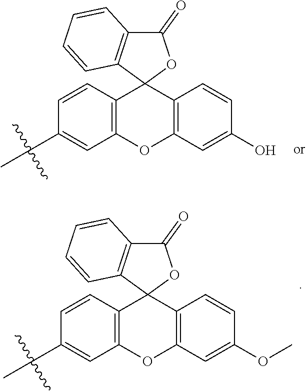 [SMILES: *c1ccc2c(c1)Oc1cc(O)ccc1C21OC(=O)c2ccccc21 ; "or" ; *c1ccc2c(c1)Oc1cc(OC)ccc1C21OC(=O)c2ccccc21 ; "."]

In some embodiments, the compound can have a structure satisfying Formula V, with some embodiments having structures satisfying Formulas VA-VD.

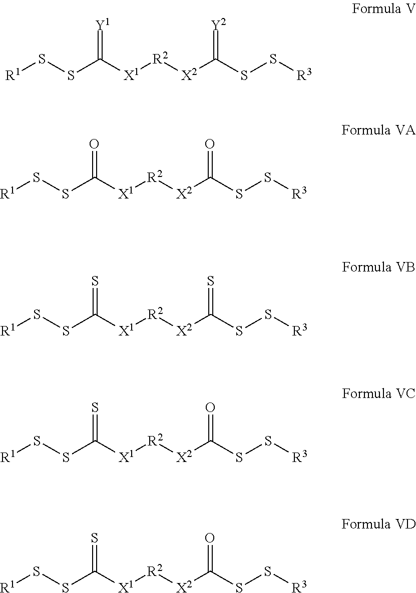

With reference to Formula V, each of Y¹ and Y² independently can be oxygen or sulfur. With reference to Formulas V and VA-VD, R¹ and R³ independently is aliphatic, heteroaliphatic, haloaliphatic, haloheteroaliphatic, aromatic, or an organic functional group; R² is aliphatic, heteraliphatic, haloaliphatic, haloheteroaliphatic, aromatic, or an organic functional group; and X¹ and X² independently are oxygen or NR⁵ (wherein R⁵ is hydrogen, aliphatic, heteroaliphatic, haloaliphatic, haloheteroaliphatic, aromatic, an organic functional group, or a combination thereof). In some embodiments of Formula V, R¹ and R³ independently is alkyl, alkenyl, alkynyl, heteroalkyl, heteroalkenyl, heteroalkynyl, heteroaryl, aryl, or combinations thereof (e.g., benzyl); R² is alkyl, alkenyl, alkynyl, heteroalkyl, heteroalkenyl, heteroalkynyl, heteroaryl, aryl, or combinations thereof. In some embodiments of Formula V, R¹ and R³ independently can be -heteroaryl-(Z) or -aryl-(Z)ₙ, and the heteroaryl and aryl ring are 6-membered rings, Z can be in the ortho, meta, or para position relative to the bond between the R¹ group and the sulfur atom (or the bond between the R³ group and the sulfur atom) depicted in Formula V. In some embodiments, n is an integer ranging from 0 to 20, such as 0 to 15, or 0 to 10, or 1 to 20, or 1 to 15, or 1 to 10 (e.g., 0, 1, 2, 3, 4, 5, 6, 7, 8, 9, 10, 11, 12, 13, 14, 15, 16, 17, 18, 19, 20). Each Z independently can be aliphatic; aromatic; heteroaliphatic (e.g., peroxy; disulfide; alkoxy; ether; thioether amino, such as —NR^{a}R^{b}, wherein R^{a} is aliphatic, heteroaliphatic, haloaliphatic, haloheteroaliphatic, aromatic, or an organic functional group and R^{b} is hydrogen, aliphatic, heteroaliphatic, haloaliphatic, haloheteroaliphatic, aromatic, or an organic functional group; or —NR^{a}R^{b}, wherein R^{a} and R^{b} are hydrogen); haloaliphatic; haloheteroaliphatic; or an organic functional group, such as, aroxy; aldehyde; acyl halide; halogen; nitro; cyano; azide; carboxyl (or carboxylate); amide; ketone; carbonate; imine; azo; carbamate; hydroxyl; thiol; sulfonyl (or sulfonate); oxime; ester; thiocyanate; thioketone; thiocarboxylic acid; thioester dithiocarboxylic acid or ester; phosphonate; phosphate; silyl ether sulfinyl; thial; or combinations thereof. In yet some additional embodiments of Formula V, R² is a detectable moiety (e.g., a fluorophore or precursor thereof). In embodiments, R² can be a xanthene derivative; cyanine or a cyanine derivative; a naphthalene derivative; coumarin or a derivative thereof; an oxadiazole derivative; an anthracene derivative; a pyrene derivative; an oxazine derivative; an acridine derivative; a fluorone dye; an isoquinoline dye; a naphthalimide compound; a chromenone dye; or a tetrapyrrole derivative (or a precursor to any such compounds). In some representative embodiments, R² is methylrhodol, 2-(2-methoxyethy)-1H-benzo[de]isoquinoline-1,3(2H)-dione, 4-methyl-2H-chromen-2-one, coumarin, naphthalimide, fluorescein, rhodamine, rhodol, Cy3, or Cy5 or a precursor thereof. In some embodiments, R² is

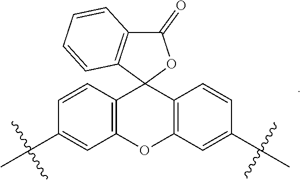 [SMILES: *c1ccc2c(c1)Oc1cc(*)ccc1C21OC(=O)c2ccccc21 ; "."]

In exemplary embodiments, the compound can be selected from:

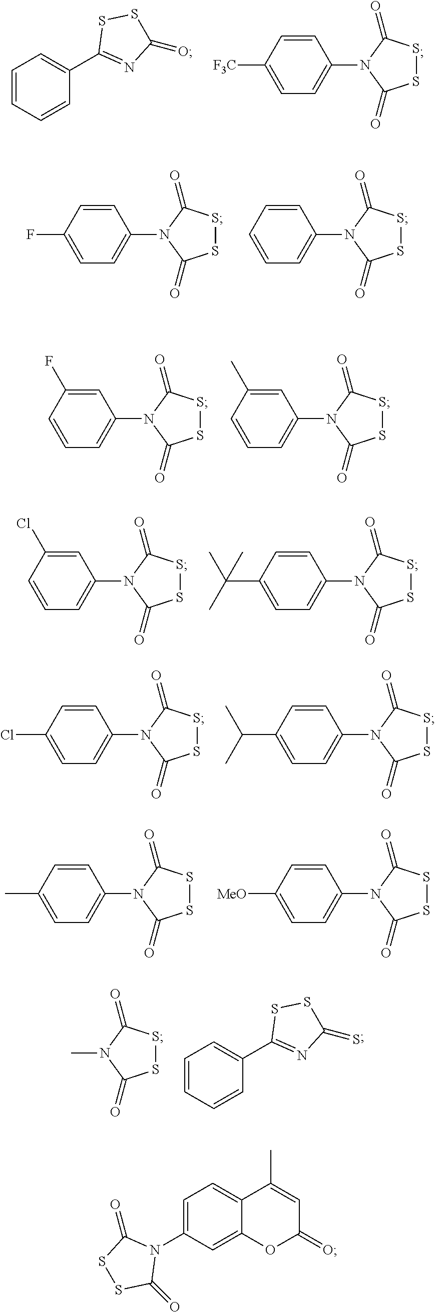

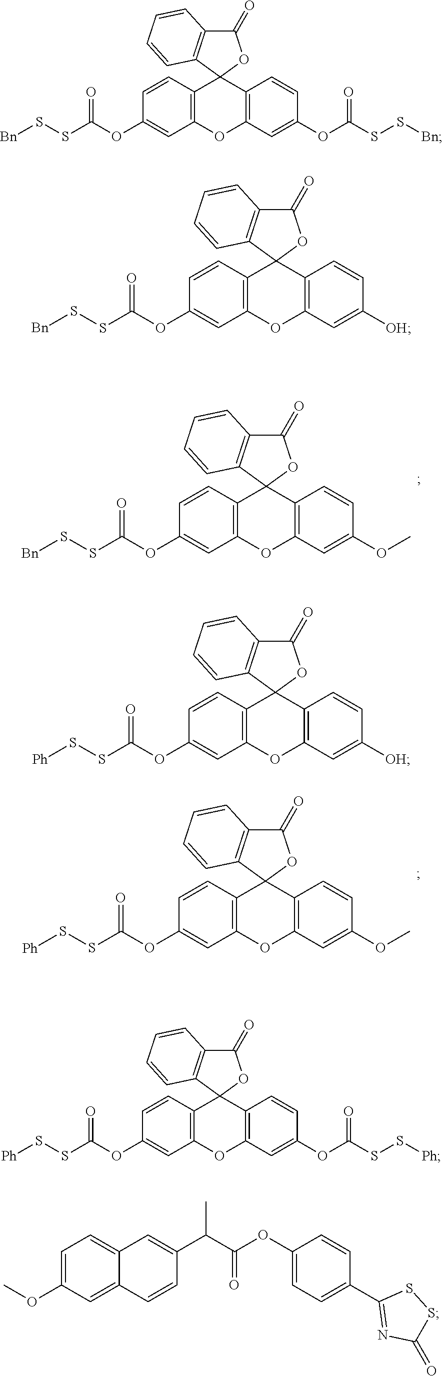

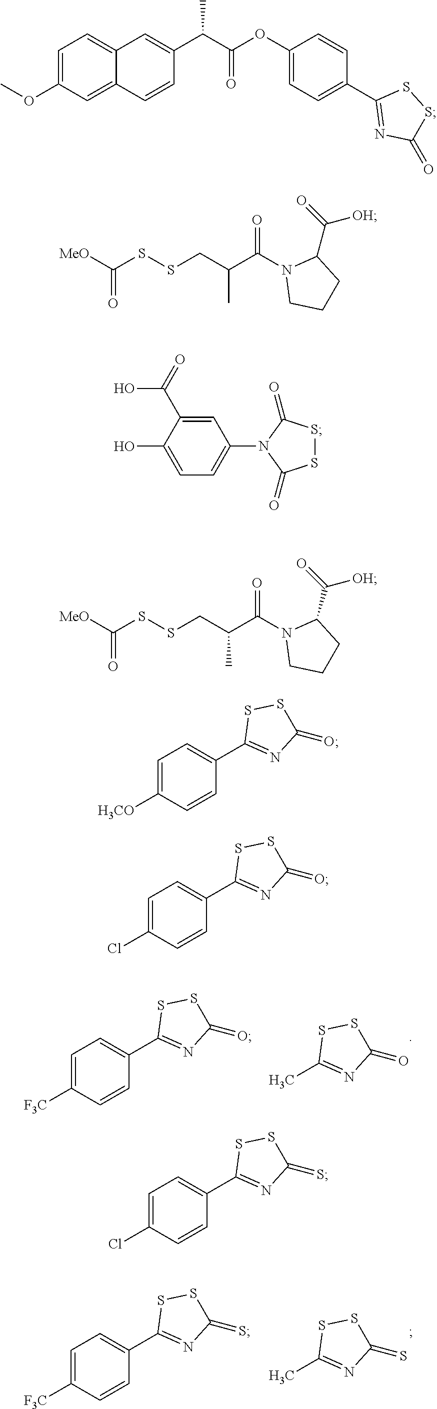

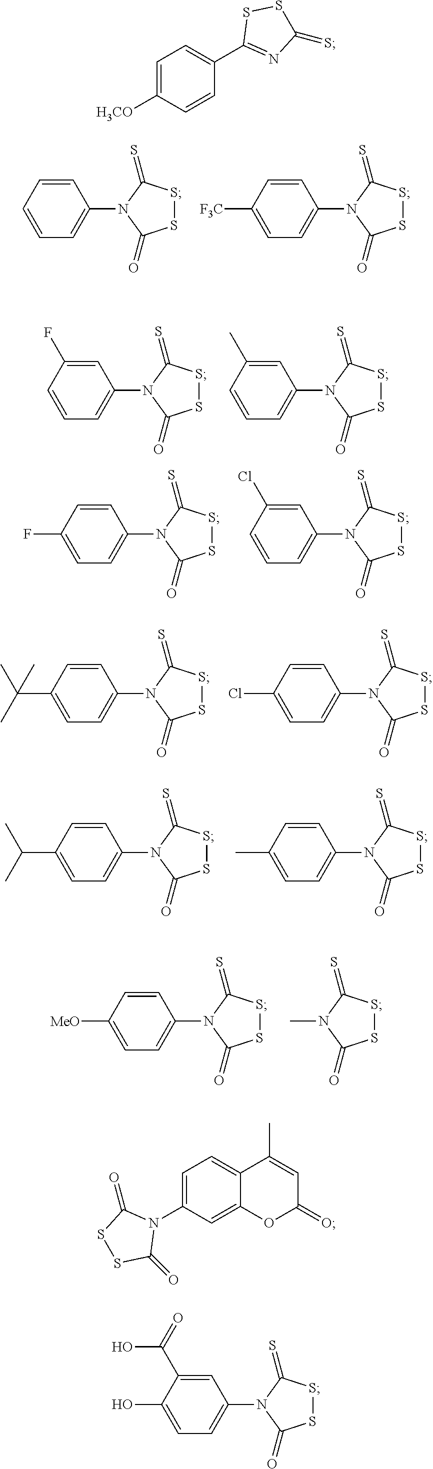

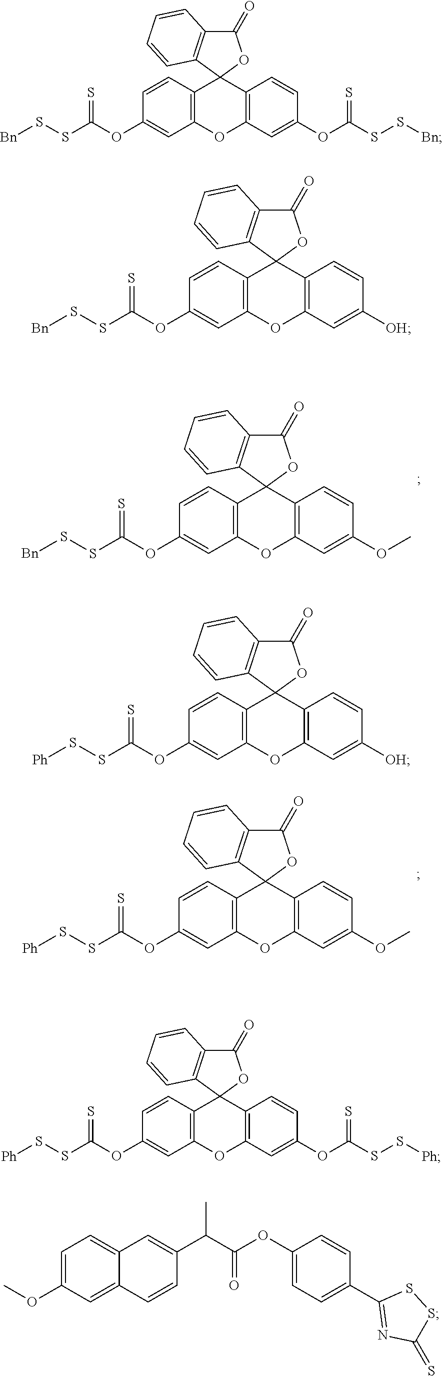

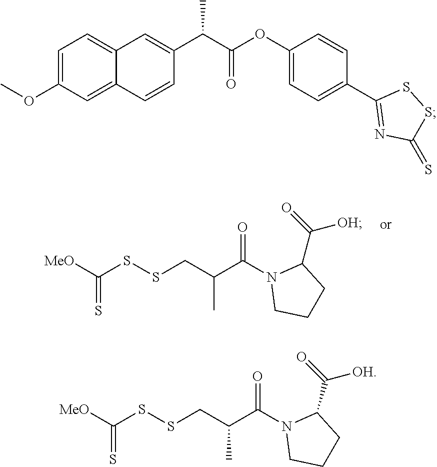

### IV. Methods of Making Compound Embodiments

Embodiments of methods for making the compound embodiments am disclosed herein. In some embodiments, certain compound embodiments can be made using a method as illustrated below in Scheme 1.

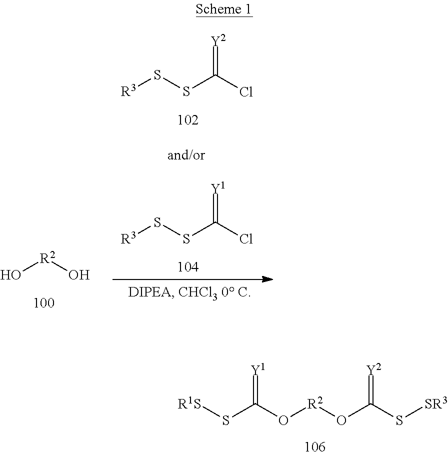

A representative method of the method of Scheme 1 is illustrated below in Scheme 2A. Scheme 2B provides an exemplary method for making a comparison compound.

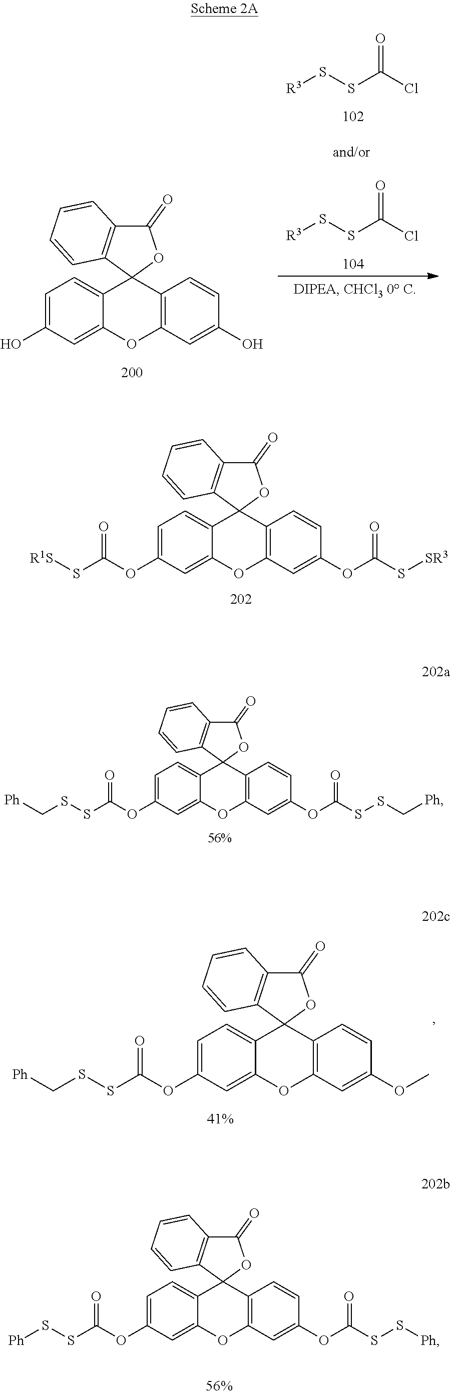

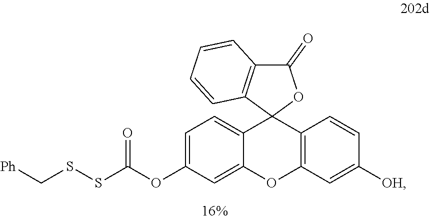 [SMILES: O=C(Oc1ccc2c(c1)Oc1cc(O)ccc1C21OC(=O)c2ccccc21)SSCc1ccccc1]

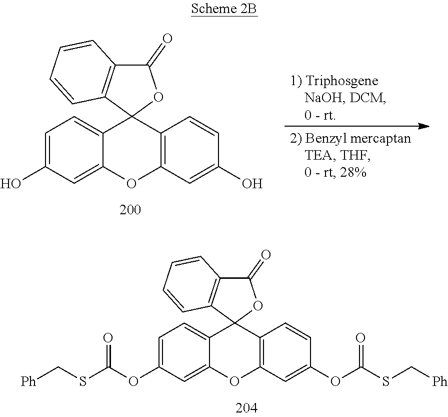 [SMILES: O=C1OC2(c3ccc(O)cc3Oc3cc(O)ccc32)c2ccccc21 ; O=C(Oc1ccc2c(c1)Oc1cc(OC(=O)SCc3ccccc3)ccc1C21OC(=O)c2ccccc21)SCc1ccccc1]

With reference to Scheme 2A, compounds 202a to 202d can be triggered by reactive compounds, such as cellular thiols (e.g., Cys and GSH), to release COS/H₂S and also provide fluorescent signals.

Control compound 204 (Scheme 2B), however, is stable toward thiol activation due to the lack of the disulfide bridge; therefore, no COS/H₂S or fluorescent signals will be generated in the presence of reactive compounds.

In yet additional embodiments, compound embodiments can be made using certain method steps illustrated below in Scheme 3.

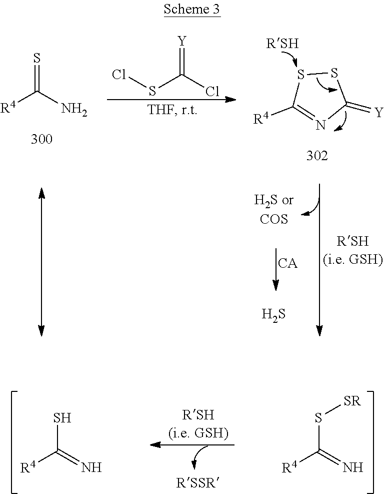

With reference to Scheme 3, compounds 302 are prepared by reacting the corresponding thioamide starting material 300 with a chlorocarbonyl sulfenyl chloride reagent. Scheme 3 further shows a proposed mechanism for how such compound embodiments can be used to release COS. As shown in Scheme 3, to activate these compounds, reactive compounds, such as cellular thiols (e.g., GSH), attack the external sulfur atom to extrude COS, which is quickly converted to H₂S by CA. The resultant disulfide intermediate further reacts with thiols to re-generate thioamide.

A representative example of a method for making compounds 302 is illustrated below in Schemes 3A, 3B, and 3C.

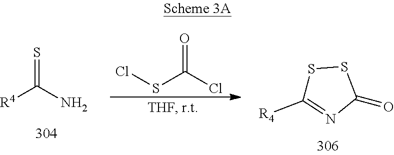

Exemplary compounds made using representative R⁴ groups are shown below.

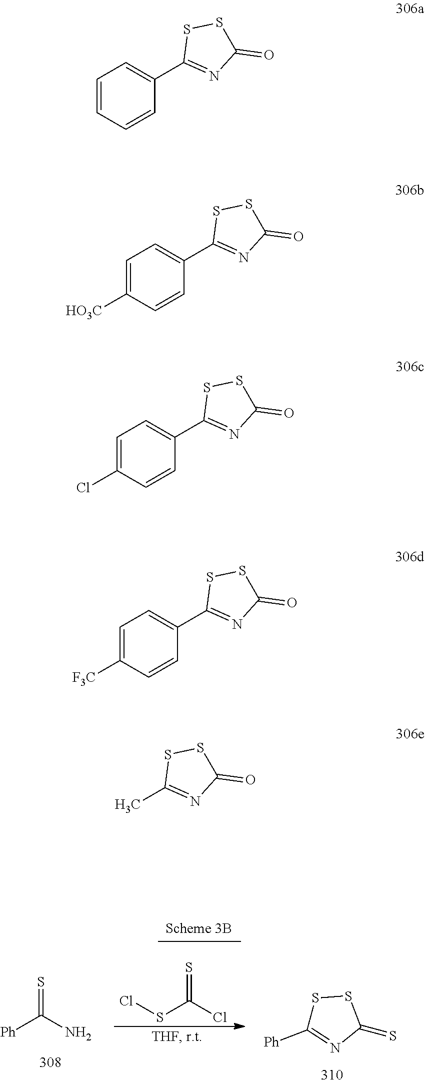

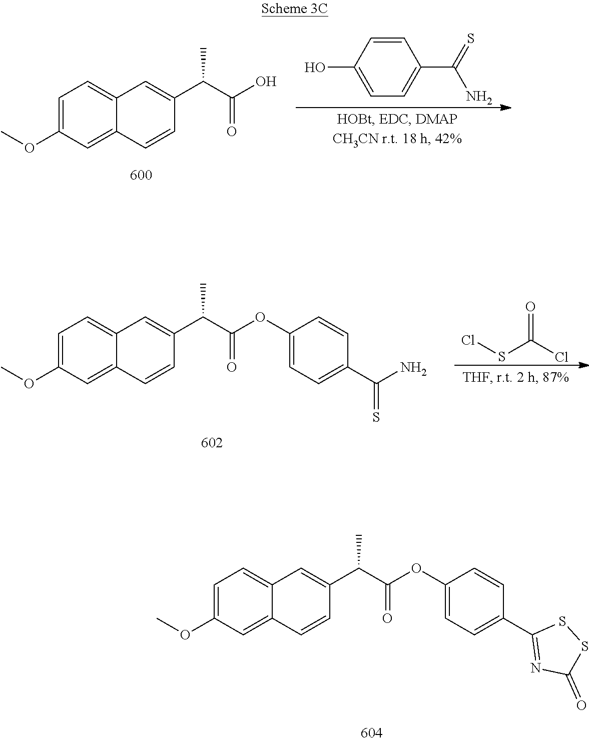 [SMILES: COc1ccc2cc([C@H](C)C(=O)O)ccc2c1 ; NC(=S)c1ccc(O)cc1 ; COc1ccc2cc([C@H](C)C(=O)Oc3ccc(C(N)=S)cc3)ccc2c1 ; O=C(Cl)SCl ; COc1ccc2cc([C@H](C)C(=O)Oc3ccc(-c4nc(=O)ss4)cc3)ccc2c1]

In yet additional embodiments, the compound embodiments can be made using the method illustrated in Scheme 4. A person of ordinary skill in the art would recognize, with the benefit of the present disclosure, that the method illustrated in Scheme 4 can be modified to use different R² groups.

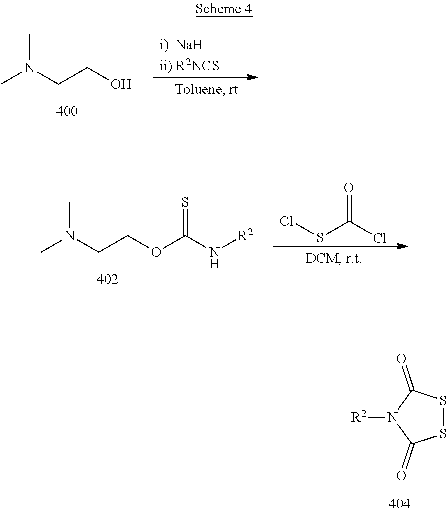

Exemplary compounds made using representative R² groups are shown below.

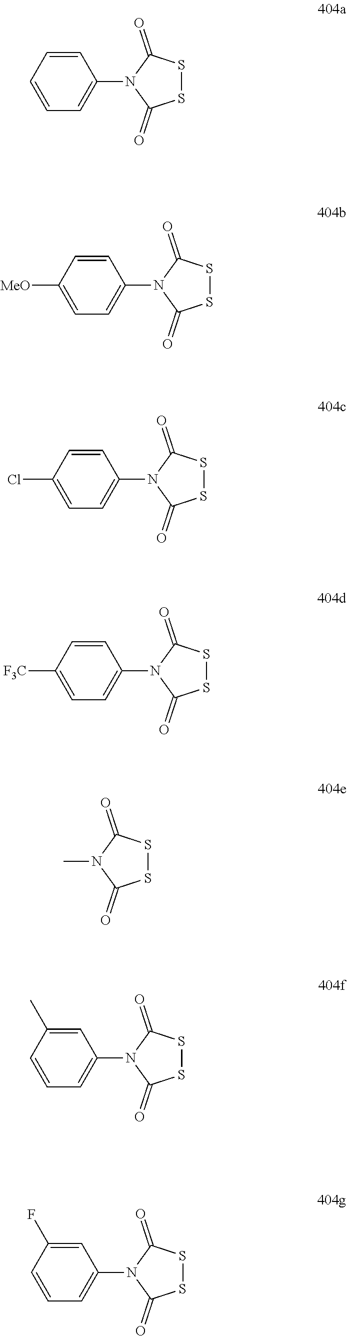

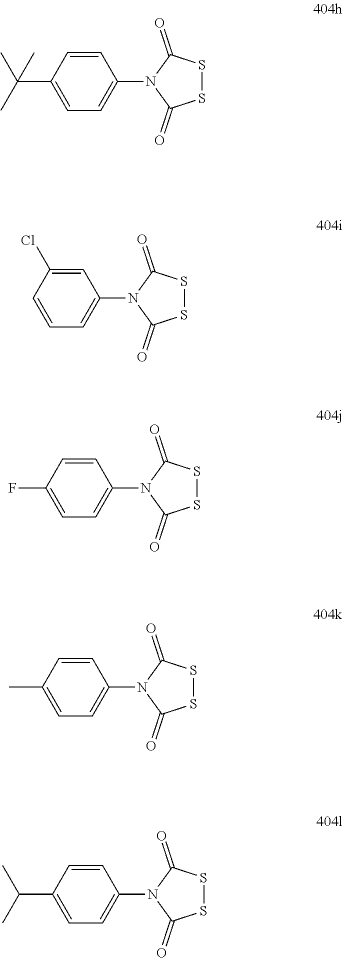 [SMILES: CC(C)(C)c1ccc(-n2c(=O)ssc2=O)cc1 ; O=c1ssc(=O)n1-c1cccc(Cl)c1 ; O=c1ssc(=O)n1-c1ccc(F)cc1 ; Cc1ccc(-n2c(=O)ssc2=O)cc1 ; CC(C)c1ccc(-n2c(=O)ssc2=O)cc1]

A representative method of that shown in Scheme 4 is provided below in Scheme 4A

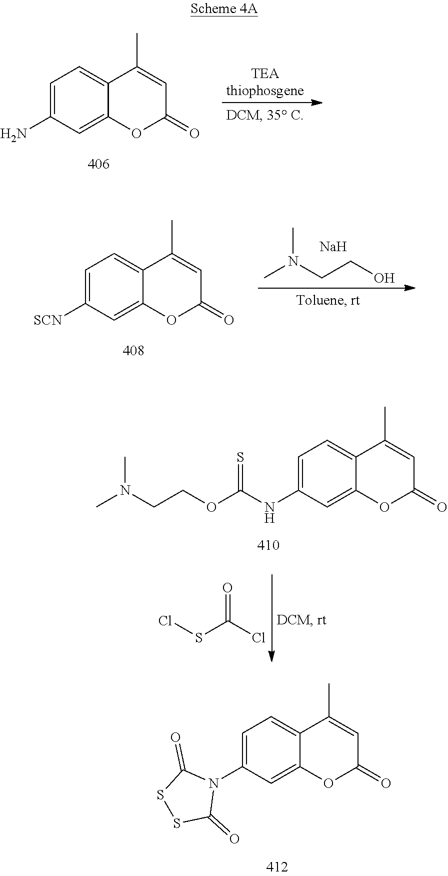

### V. Methods of Using Compound Embodiments

In some embodiments, the compound embodiments described herein can be used to generate COS and/or H₂S. In yet additional embodiments, the compound can also release an active agent upon COS and/or H₂S release. In yet additional embodiments, the compound can also exhibit a detectable signal upon COS and/or H₂S release. In such embodiments, the compound typically comprises a built-in signal generating moiety, such as a fluorophore, a quantum dot, or a member of a specific binding pair. The compound embodiments disclosed herein are stable in aqueous media and do not spontaneously release COS/H₂S or give off fluorescent signals. However, in the presence of reactive compounds, such as thiol-containing compounds (e.g., cellular thiols), the disclosed compound embodiments are activated through reaction of the reactive compound with the disulfide functional group included in the compound. The resultant intermediate releases COS, which is converted to H₂S by carbonic anhydrase (CA), with a concomitant fluorescence turn on in some embodiments. Compound activation can be observed in cellular environments and the released H₂S exhibits potent anti-inflammatory activity against LPS-induced inflammation. A schematic illustration of a representative pathway is shown in FIG. 1.

In embodiments described herein, the compound, or any composition thereof, can be used to generate COS and/or H₂S and thus can be used to deliver COS and/or H₂S to a subject or sample. In some embodiments, the compound can comprise an active agent (e.g., a therapeutic agent) and thus also can be used to deliver the therapeutic agent to a subject simultaneously (or substantially simultaneously) with COS and/or H₂S release. The compound embodiments can be used in in vivo, in vitro, or ex vivo methods to increase COS and/or H₂S concentration and/or COS and/or H₂S activity in a sample or a subject and also to treat a subject by delivering a therapeutic agent to the subject.

In particular disclosed embodiments, the method can comprise exposing a subject or a sample to a compound embodiment or a composition thereof. In some embodiments, the method is an in vitro method and it comprises exposing a sample, such as a biological sample obtained from a subject (or other samples), to the compound or a composition thereof. In some embodiments, the method is an in vivo method and it comprises exposing a subject, such as a human or other animal, to the compound or a composition thereof (such as a pharmaceutical composition). In some embodiments, the subject or the sample can be exposed to an amount of the compound that is sufficient to increase the amount of COS and/or H₂S in the subject or sample to a certain level. For example, in subjects or samples that are determined to have deficient amounts of COS and/or H₂S, the compound can be administered at a concentration sufficient to increase the H₂S concentration back to a normally accepted level. What constitutes a “normally accepted level” can depend on the type of subject or sample (e.g., cell or tissue types involved), but could be determined by a person of ordinary skill in the art with the benefit of this disclosure. In some embodiments, the “normally accepted level” can exist in the nanomolar to low micromolar range.

Dosage amounts, such as therapeutically effective amounts, of the compound embodiments typically are selected to be amounts that will deliver H₂S and/or a therapeutic agent, wherein such compounds are individually delivered in amounts ranging from greater than 0 mg/kg/day (such as 0.0001 mg/kg/day, 0.001 mg/kg/day, or 0.01 mg/kg/day) to 100 mg/kg/day. In embodiments where the compound is administered as a pharmaceutical composition, the amount of the compound in the composition can be an amount sufficient to deliver H₂S and/or a therapeutic agent (individually) in amounts ranging from greater than 0 mg/kg/day (such as 0.0001 mg/kg/day, 0.001 mg/kg/day, or 0.01 mg/kg/day) to 100 mg/kg/day.

In some embodiments, the method can further comprise exposing the subject or the sample to a reactive compound that facilitates release of COS and/or H₂S. In some embodiments, however, the reactive compound can inherently be present in the subject or the sample. In embodiments where the reactive compound is added to the subject or sample, it can be administered by any suitable means (e.g., immersing the sample in a solution comprising the reactive compound; or by oral administration, parenteral administration, or the like).

In some embodiments, the method can further comprise detecting and/or measuring a detectable signal produced after exposing the sample or the subject to the compound, and/or after exposing the sample or the subject to a reactive compound. In some embodiments, the detectable signal is produced by a detectable moiety. Particular embodiments comprise a moiety that fluoresces upon reaction of the compound with a thiol-containing compound. In some embodiments, detecting a detectable signal can comprise visualizing a color, fluorescent, and/or phosphorescent change in a sample (e.g., by using the naked eye or by using a fluorescent lamp). In some embodiments, detecting and/or measuring a detectable signal can comprise using a measurement technique, such as using spectroscopic methods (e.g., UV-visible spectroscopy, fluorescence spectroscopy, phosphorescence spectroscopy, or the like), a fluorescent microscope, a fluorescence scanner, or a flow cytometer to observe and/or quantify the detectable signal.

A representative method for using the compound embodiments described herein is illustrated in Scheme 7. As illustrated in Scheme 7, the compound is activated by a thiol-induced disulfide reduction. The resultant intermediate then undergoes dethiocarboxylation to release COS, which is converted to H₂S by the ubiquitous enzyme carbonic anhydrase (CA). In the representative embodiment illustrated in Scheme 7, the compound releases COS and the remaining skeleton of the compound concomitantly exhibits fluorescence, which enables a real-time tracking of COS/H₂S delivery.

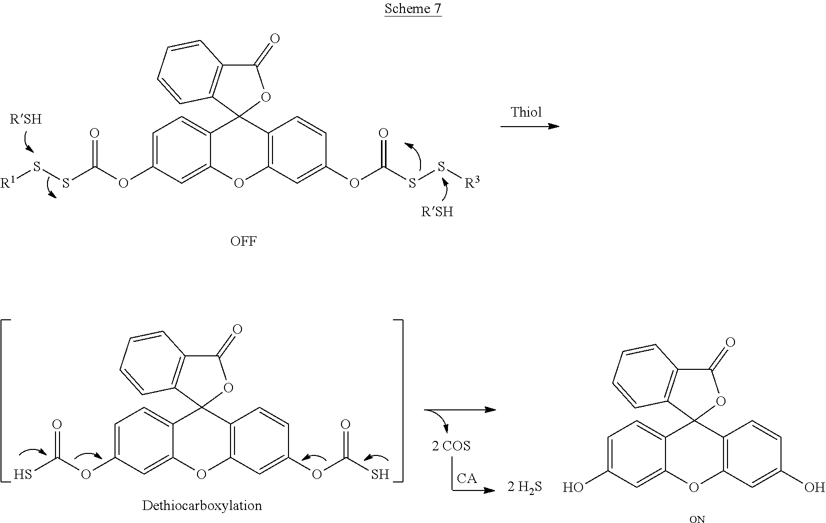

Another representative method for using the compound embodiments described herein is illustrated in Scheme 8.

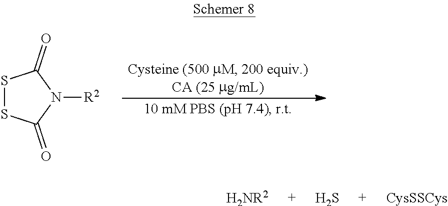

In additional embodiments, the compound embodiments described herein can be used to release active agents, such as therapeutic drugs. In some embodiments, compounds of Formula I can comprise an R group or an R′ group that comprises the active agent, such as a drug molecule. Although these compounds do not result in a fluorescence response, they release the drug during the thiol-triggered release process. Two representative examples of such compound embodiments are shown below in which the payload is an ACE inhibitor drug (Compound A) or is a naproxen molecule (Compound B).

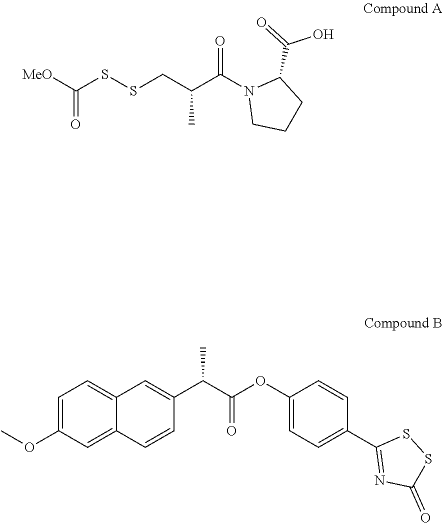 [SMILES: COC(=O)SSC[C@@H](C)C(=O)N1CCC[C@H]1C(=O)O ; COc1ccc2cc([C@H](C)C(=O)Oc3ccc(-c4nc(=O)ss4)cc3)ccc2c1]

Composition embodiments comprising a compound of the present disclosure also are disclosed herein. In some embodiments, the composition comprises a compound embodiment, or a plurality thereof. In some embodiments, the composition can further comprise water, a buffer, or any combination thereof. In some embodiments, the composition can be a pharmaceutical composition that comprises a compound and one or more pharmaceutically acceptable excipients, water, a pharmaceutically acceptable buffer, a separate therapeutic agent, or any combinations thereof. In some embodiments, the pharmaceutical composition comprises a compound comprising a therapeutic agent, such as certain compound embodiments described above.

### VI. Overview of Several Embodiments

Disclosed herein are embodiments of a compound having a structure satisfying Formula I

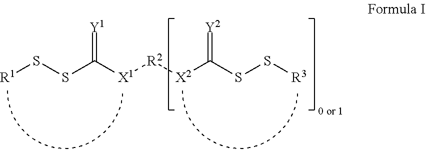

wherein: R¹ and R³ independently are aliphatic, heteroaliphatic, haloaliphatic, haloheteroaliphatic, aromatic, an organic functional group, or any combination thereof; or R¹ and X¹, together, provide a 5-membered ring and/or R³ and X², together, provide a 5-membered ring; X¹ and X² independently are oxygen, nitrogen, or NR⁵, wherein R⁵ is hydrogen, aliphatic, heteroaliphatic, haloaliphatic, haloheteroaliphatic, aromatic, an organic functional group, or a combination thereof; each of Y¹ and Y² independently is oxygen or sulfur; and R², if present, is hydrogen, aliphatic, heteroaliphatic, haloaliphatic, haloheteroaliphatic, aromatic, an organic functional group, or a combination thereof.

In some embodiments, the compound has a structure satisfying Formula V

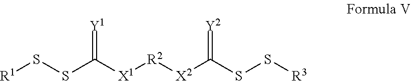

wherein R¹ and R³ independently are aromatic or an organic functional group; X¹ and X² independently are oxygen or NR⁵; and R² is aromatic or heteroaliphatic.

In any or all of the above embodiments, R¹ and R³ independently are benzyl or phenyl and R² is heteroaryl or heterocyclic.

In any or all of the above embodiments, X¹ and X² are oxygen and R² is

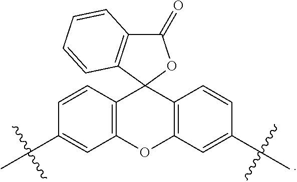 [SMILES: *c1ccc2c(c1)Oc1cc(*)ccc1C21OC(=O)c2ccccc21]

In some embodiments, R¹ is C(O) and X¹ is N, and wherein R¹ and X¹, together, provide a 5-membered ring wherein R¹ is bound to X¹ via a single bond, thereby providing a compound having a structure satisfying Formula III

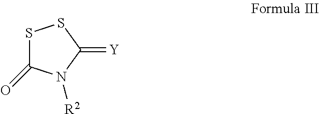

wherein R² is aliphatic, heteroaliphatic, haloaliphatic, haloheteroaliphatic, aromatic, an organic functional group, or a combination thereof.

In any or all of the above embodiments, R² is alkyl, heteroaryl, aryl, or a heterocyclic.

In any or all of the above embodiments, R² is -heteroaryl-(Z)ₙ or -aryl-(Z)ₙ, wherein each Z is a substituent other than hydrogen and n is an integer ranging from 0 to 20. In some embodiments, each Z independently is aliphatic; aromatic; heteroaliphatic; haloaliphatic; haloheteroaliphatic; an organic functional group; or any combination thereof.

In any or all of the above embodiments, the organic functional group is selected from aroxy; aldehyde; acyl halide; halogen; nitro; cyano; azide; carboxyl (or carboxylate); amide; ketone; carbonate; imine; azo; carbamate; hydroxyl; thiol; sulfonyl (or sulfonate); oxime; ester; thiocyanate; thioketone; thiocarboxylic acid; thioester; dithiocarboxylic acid or ester; phosphonate; phosphate; silyl ether; sulfinyl; thial; or combinations thereof; and/or wherein the heteroaliphatic group is selected from peroxy; disulfide; alkoxy; ether; thioether; or amino.

In some embodiments, the compound has a structure satisfying Formula IV

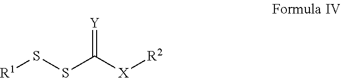

wherein R¹ is aliphatic, heteroaliphatic, haloaliphatic, haloheteroaliphatic, aromatic, an organic functional group, or any combination thereof; and R² is hydrogen, aliphatic, heteroaliphatic, haloaliphatic, haloheteroaliphatic, aromatic, an organic functional group, or a combination thereof.

In any or all of the above embodiments, R¹ is phenyl or benzyl; X is oxygen; and wherein R² is

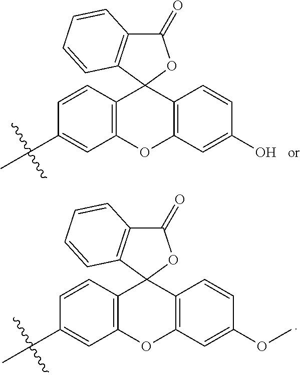 [SMILES: *c1ccc2c(c1)Oc1cc(O)ccc1C21OC(=O)c2ccccc21 ; *c1ccc2c(c1)Oc1cc(OC)ccc1C21OC(=O)c2ccccc21]

In any or all of the above embodiments, R¹ is

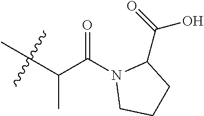 [SMILES: *C(C)C(=O)N1CCCC1C(=O)O]

and, X is oxygen, and R² is alkyl.

In some embodiments, R¹ is CR⁴ and X¹ is nitrogen and R¹ and X¹, together, provide a 5-membered ring wherein R¹ is bound to X¹ via a double bond, thereby providing a compound having a structure satisfying Formula II

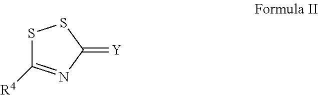

wherein R⁴ is aliphatic, heteroaliphatic, haloaliphatic, haloheteroaliphatic, aromatic, an active agent, an organic functional group, or any combination thereof.

In any or all of the above embodiments, R⁴ is -heteroaryl-(Z)ₙ or -aryl-(Z)ₙ, wherein each Z is a substituent other than hydrogen and n is an integer ranging from 0 to 20. In some embodiments, each Z independently is aliphatic; aromatic; heteroaliphatic; haloaliphatic; haloheteroaliphatic; an active agent; an organic functional group; or any combination thereof.

In any or all of the above embodiments, the organic functional group is selected from aroxy; aldehyde; acyl halide; halogen; nitro; cyano; azide; carboxyl (or carboxylate); amide; ketone; carbonate; imine; azo; carbamate; hydroxyl; thiol; sulfonyl (or sulfonate); oxime; ester; thiocyanate; thioketone; thiocarboxylic acid; thioester; dithiocarboxylic acid or ester; phosphonate; phosphate; silyl ether; sulfinyl; thial; or combinations thereof; and/or wherein the heteroaliphatic group is selected from peroxy; disulfide; alkoxy; ether; thioether; or amino.

In some embodiments, the has structure satisfying any one or more of Formulas IIA-VA

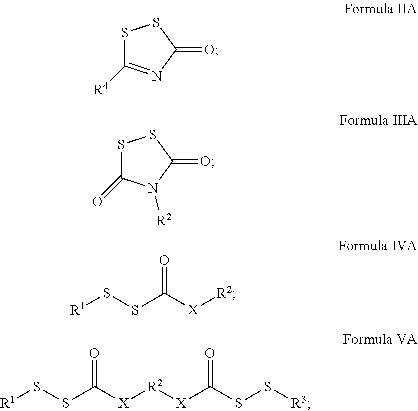

wherein: R¹ and R³ independently are aliphatic, heteroaliphatic, haloaliphatic, haloheteroaliphatic, aromatic, or any combination thereof; or R¹ (and/or R³) can join with an X group to provide a ring; each X independently is oxygen or NR⁵, wherein R⁵ is hydrogen, aliphatic, heteroaliphatic, aromatic, or any combination thereof; R² is hydrogen, aliphatic, heteroaliphatic, haloaliphatic, haloheteroaliphatic, aromatic, or any combination thereof when the compound has a structure satisfying Formula IV; or R² is aliphatic, heteroaliphatic, haloaliphatic, haloheteroaliphatic, aromatic, or any combination thereof when the compound has a structure satisfying Formulas III or V; and R⁴ is aliphatic, heteroaliphatic, aromatic, or any combination thereof.

In any or all of the above embodiments, the compound is selected from any of the representative species structures provided herein.

Also disclosed herein are embodiments of a pharmaceutical composition, comprising a compound according to any or all of the above embodiments and further comprising a pharmaceutically acceptable excipient.

Also disclosed herein are embodiments of a method comprising exposing a sample or a subject to a compound according to any or all of the above embodiments, or a pharmaceutical composition thereof.

In some embodiments, the method further comprises analyzing the sample or the subject to detect a reaction between the compound and a thiol-containing compound that is inherently present in the subject or the sample or that is added to the subject or the sample, wherein the reaction produces a detectable signal, COS, H₂S, or a combination thereof. In some embodiments, analyzing comprises detecting and/or measuring a fluorescence change, or a change in concentration of H₂S, or any combination thereof.

In any or all of the above embodiments, the method further comprises measuring an amount of H₂S released from the compound.

In any or all of the above embodiments, the sample is a biological sample selected from a cell, tissue, and/or bodily fluid.

In any or all of the above embodiments, the method comprises exposing a subject that has or is at risk of developing a disease associated with H₂S deficiency or H₂S misregulation.

In any or all of the above embodiments, the disease is a cardiovascular disease, diabetes, inflammation, a neurological disease, cancer, a disease involving insufficient wound healing, erectile dysfunction, or any combinations thereof. In some embodiments, the cardiovascular disease is heart failure, myocardial reperfusion injury, atherosclerosis, hypertension, hypertrophy, or any combinations thereof.

### VII. Examples

Methods and Materials—Reagents were purchased from Sigma-Aldrich, Tokyo Chemical Industry (TCI), Fisher Scientific, Combi-Blocks, and VWR and used directly as received. Silica gel (SiliaFlash F60, Silicycle, 230-400 mesh) was used for column chromatography. Deuterated solvents were purchased from Cambridge Isotope Laboratories (Tewksbury, Mass., USA). ¹H and ¹³C NMR spectra were recorded on Bruker 500 MHz NMR instruments at the indicated frequencies. Chemical shifts are reported in ppm relative to residual protic solvent resonances. Mass spectrometric measurements were performed by the University of Illinois, Urbana Champaign MS facility, or on a Xevo Waters ESI LC/MS instrument. Fluorescein intensity was measured using a Quanta Master 40 spectrofluorometer (Photon Technology International) and methylene blue absorbance was monitored by a Cary 60 UV-Vis spectrometer.

Compounds 1, 2, and C-Az were synthesized by following the literature reports. HeLa cells and RAW 264.7 cells were purchased from ATCC (Manassas, Va., USA). Cell imaging experiments were performed on a Leica DMi8 fluorescence microscope, equipped with an Andor Zyla 4.2+ sCMOS detector. NO₂— levels were obtained by using a Griess Reagent kit (Thermo Fisher Scientific) and the absorbance at 548 nm was measured by using a microplate reader (Tecan Spark 20M).

Representative Measurement of Fluorescence Intensity of Compound Embodiments—A freshly prepared compound 202a stock solution (3.00 μL, 10.0 mM in DMSO) was added to 3.00 mL of PBS (Ph 7.40, 10.0 Mm) containing CA (25.0 μg/mL) in a quartz fluorescence cuvette. A Cys stock solution (10.0 mM) was then added to reach the desired working concentration. The reaction solution was excited at 490 nm and the fluorescence intensity (500-650 nm) was measured and recorded using a Quanta Master 40 spectrofluorometer.

RepresentatIve Measurement of HSS Release from Compound Embodiments by MB Assay—A compound 202a stock solution (20.0 μL, 10.0 mM in DMSO) was added to 20.0 mL of PBS (pH 7.40, 10.0 mM) containing CA (25.0 μg/mL) in a 20-mL scintillation vial. A Cys stock solution (20.0 μL, 100 mM) was then added. Next, 300 μL aliquots of the reaction mixture were transferred to UV cuvettes containing 300 μL of MB cocktail (60.0 μL Zn(OAc)₂ (1.00% w/v), 120 μL FeCl₃ (30.0 mM in 1.20 M HCl), and 120 μL N,N-dimethyl-p-phenylene diamine (20.0 mM in 7.20 M HC)) at different time points. The absorbance at 670 nm was then measured after 1 hour and was converted to H₂S concentration by using an H₂S calibration curve.

Representative Procedure for Evaluating Selectivity of Compound EmbodIments to Cellular RSONs—To 3.00 mL of PBS was added a stock solution of compound 202a (3.00 μL, 10.0 mM in DMSO), followed by the addition of RSON stock solution (30.0 μL, 10.0 mM in H₂O). After 2 hours of incubation at room temperature, the solution was excited at 490 nm and the fluorescence intensity (500-650 nm) was measured and recorded by using a Quanta Master 40 spectrofluorometer. For the NEM-pretreated group, NEM (30.0 μL, 1.00 M) was added to PBS (3.00 mL) containing CA (25.0 μg/mL) and Cys (100 μM). The solution was then incubated for 1 hour before adding the compound 202a stock solution (3.00 μL, 10.0 mM in DMSO).

Representative Procedure for Cell Culture and Cellular Imaging of H₂S Delivery from Compound Embodiments—HeLa cells were cultured in high glucose Dulbecco's modified Eagle's medium (DMEM) supplemented with 10% fetal bovine serum (FBS) and 1% penicillin/streptomycin at 37° C. under 5% CO₂. HeLa cells were then plated in poly-D-lysine coated plates (MatTek) containing 2.00 mL of DMEM and incubated at 37° C. under 5% CO₂ for 24 hours. The confluent cells were washed with PBS and then co-incubated with NucRed nuclear dye (2 drops), C7-Az (50.0 μM), and compound 202a (50.0 μM) for 30 minutes. Prior to imaging, the cells were washed with PBS and bathed in 2.00 mL of PBS. Cell imaging was performed on a Leica DMi8 fluorescent microscope.

Representative Procedure for Determining Anti-Inflammatory Activities of Compound Embodiments and Control Compounds—Macrophage RAW 264.7 cells were seeded in a 24-well plate (5×105 cells/well) containing 0.500 mL of DMEM and incubated at 37° C. under 5% CO₂ for 24 hours. The confluent cells were washed with PBS and incubated with either compound 202a (0-25.0 μM), or GYY4137 (25.0 μM) at 37° C. for 2 hours. Compounds were then removed by washing cells with PBS and these pretreated cells were incubated in FBS-free DMEM containing LPS (0.500 μg/mL) for 24 hours. NO₂— levels were measured by using a Griess Reagent Kit.

Representative Procedure for Making Compound Embodiments—A fluorescein starting material (1.00 equiv.) was added to CHCl₃ containing 102 or 104 (3.00 equiv.). After stirring the reaction mixture at 0° C. for 5 minutes, DIPEA (3.00 equiv.) was added slowly. The reaction solution was stirred at room temperature until the completion of the reaction as indicated by TLC (usually less than 2 h). The reaction was then quenched by adding brine (25 mL), and the aqueous solution was extracted with ethyl acetate (3×15 mL). The organic layers were combined, dried over MgSO₄, and evaporated under vacuum. The product was isolated after purification by column chromatography.

### Example 1

Compound 202a was prepared by reacting fluorescein (332 mg, 1.00 mmol) with 102 (654 mg, 3.00 mmol) in the presence of DIPEA (390 mg, 3.00 mmol) using the general synthetic procedure described above. Compound 202a was isolated as yellow solid by column chromatography using ethyl acetate/hexanes (1/3, v/v, R_{f}=0.31) as the eluent (390 mg, 56% yield). ¹H NMR (500 MHz, DMSO-d₆) δ (ppm): 8.09 (d, J=5.0 Hz, 1H), 7.85 (t, J=5.0 Hz, 1H), 7.79 (t, J=5.0 Hz, 1H), 7.38 (m, 13H), 7.01 (d, J=10.0 Hz, 2H), 6.95 (d, J=10.0 Hz, 2H), 4.18 (s, 4H). ¹³C{¹H} NMR (125 MHz, DMSO-d₆) δ (ppm): 168.8, 167.8, 152.6, 151.2, 136.5, 131.1, 130.1, 130.0, 129.0, 128.2, 125.7, 125.6, 124.6, 118.4, 117.5, 110.6, 81.1, 42.4. IR (cm⁻¹): 2981, 1744, 1608, 1408, 1420, 1237, 1143, 1107, 1060, 988, 881, 751. HRMS m/z [M+H]+calcd. For [C₃₆H₂₅O₇S₄]⁺ 697.0483; found 697.0474.

### Example 2

Compound 202b was prepared by reacting fluorescein (93.0 mg, 0.280 mmol) with 104 (171 mg, 0.840 mmol) in the presence of DIPEA (109 mg, 0.840 mmol) using the general synthetic procedure described above. Compound 202b was isolated as white solid by column chromatography using ethyl acetate/hexanes (1/2, v/v, R_{f}=0.49) as the eluent (105 mg, 56% yield). ¹H NMR (500 MHz, DMSO-d₆) δ (ppm): 8.08 (d, J=5.0 Hz, 1H), 7.83 (t, J=5.0 Hz, 1H), 7.78 (t, J=5.0 Hz, 1H), 7.65 (d, J=10.0 Hz, 4H), 7.55 (s, 2H), 7.43 (m, 7H), 7.14 (d, J=5.0 Hz, 2H), 6.96 (d, J=5.0 Hz, 2H). ¹³C{¹H} NMR (125 MHz, DMSO-d) δ (ppm): 168.8, 167.7, 152.6, 151.2, 136.6, 131.1, 130.4, 130.1, 130.0, 129.5, 125.7, 125.6, 124.6, 118.5, 117.7, 110.8, 81.0. IR (cm⁻¹): 3057, 2923, 1747, 1607, 1581, 1489, 1419, 1327, 1285, 1235, 1219, 1142, 1107, 1061, 881, 685. HRMS m/z [M+H]⁺ calcd. For [C₃₄H₂₁O₇S₄]⁺ 669.0170; found 669.0173.

### Example 3

Compound 202c was prepared by reacting 3-O-methylfluorescein (69.0 mg, 0.207 mmol) with 102 (136 mg, 0.623 mmol) in the presence of DIPEA (81.0 mg, 0.623 mmol) using the general synthetic procedure described above. Compound 202c was isolated as white solid by column chromatography using ethyl acetate/hexanes (1/1, v/v, R_{f}=0.64) as the eluent (43.0 mg, 41% yield). ¹H NMR (500 MHz, DMSO-da) δ (ppm): 8.06 (d, J=10.0 Hz, 1H), 7.83 (t, J=5.0 Hz, 1H), 7.77 (t, J=5.0 Hz, 1H), 7.36 (m, 7H), 6.98 (d, J=10.0 Hz, 2H), 6.91 (d, J=10.0 Hz, 2H), 6.77 (d, J=10.0 Hz, 1H), 6.73 (d, J=10.0 Hz, 1H), 4.18 (s, 2H), 3.84 (s, 3H). ¹³C{¹H} NMR (125 MHz, DMSO-d₆) δ (ppm): 168.9, 167.8, 161.7, 152.7, 152.4, 151.9, 151.5, 136.5, 136.4, 130.9, 130.1, 130.0, 129.5, 129.0, 128.2, 126.1, 125.4, 124.5, 118.0, 117.8, 113.0, 110.9, 110.4, 101.3, 81.9, 56.2, 42.4. IR (cm⁻¹): 2981, 1747, 1607, 1491, 1420, 1241, 1220, 1144, 1103, 1060, 986, 874. HRMS m/z [M+H]⁺ calcd. For [C₂₉H₂₁O₆S₂]⁺ 529.0780; found 529.0779.

### Example 4

Compound 202d was prepared by reacting fluorescein (166 mg, 0.500 mmol) with 102 (22.0 mg, 0.100 mmol) in the presence of DIPEA (13.0 mg, 0.100 mmol) using the general synthetic procedure described above. Compound 202d was isolated as yellow solid by column chromatography using ethyl acetate/hexanes (1/1, v/v, R_{f}=0.52) as the eluent (8.00 mg, 16% yield). ¹H NMR (500 MHz, DMSO-d₆) δ (ppm): 10.23 (s, 1H), 8.04 (d, J=10.0 Hz, 1H), 7.83 (t, J=5.0 Hz, 1H), 7.76 (t, J=5.0 Hz, 1H), 7.35 (m, 7H), 6.95 (d, J=10.0 Hz, 1H), 6.87 (d, J=10.0 Hz, 1H), 6.74 (s, 1H), 6.62 (s, 2H), 4.18 (s, 2H). ¹³C{¹H}NMR (125 MHz, DMSO-d₆) δ (ppm): 169.0, 167.8, 160.2, 152.7, 152.4, 151.9, 151.6, 136.5, 136.3, 130.9, 130.1, 129.9, 129.6, 129.0, 128.2, 126.2, 125.3, 124.5, 118.0, 117.8, 113.7, 110.5, 109.5, 102.7, 82.2, 42.4. IR (cm⁻¹): 3057, 2923, 1747, 1607, 1581, 1489, 1419, 1285, 1219, 1142, 1107, 1061. HRMS m/z [M+H]⁺ calcd. For [C₂₈H₁₉O₆S₂]⁺ 515.0623; found 515.0620.

### Example 5

Control compound 204. The fluorescein starting material (33.0 mg, 0.100 mmol) was combined with triethyl amine (40.0 mg, 0.400 mmol) in anhydrous THF. After stirring the reaction mixture at 0° C. for 10 minutes, benzyl chlorothioformate (75.0 mg, 0.400 mmol) was added slowly. The reaction solution was stirred at room temperature for 2 hours. The reaction was then quenched by adding brine (25 mL), and the aqueous solution was extracted with ethyl acetate (3×15 mL). The organic layers were combined, dried over MgSO₄, and evaporated under vacuum. Control compound 204 was isolated as white solid by column chromatography using ethyl acetate/hexanes (1/2, v/v, R=0.50) as the eluent (57.0 mg, 91% yield). ¹H NMR (500 MHz, CDCl₃) δ (ppm): 8.07 (d, J=5.0 Hz, 1H), 7.68 (m, 2H), 7.38 (m, 8H), 7.31 (m, 2H), 7.19 (m, 3H), 6.88 (m, 4H), 4.22 (s, 4H). ¹³C{¹H} NMR (125 MHz, CDCl₃) δ (ppm): 169.5, 169.0, 152.9, 152.4, 151.5, 136.4, 135.3, 130.1, 129.1, 129.0, 128.8, 127.8, 126.0, 125.3, 124.0, 117.4, 116.8, 110.2, 81.4, 35.8. IR (cm⁻¹): 2981, 1763, 1716, 1607, 1490, 1419, 1237, 1144, 1062, 989, 752, 687. HRMS m/z [M+H]J calcd. For [C₃₆H₂₅O₇S₂]⁺ 633.1042; found 633.1050.

### Example 6

In this example, the spectroscopic properties in PBS buffer (pH 7.4, 10 mM) of compounds 202a-202d, and control compound 204. Compounds 202a-202c and control compound 204 were not absorptive in the visible region and are all these compounds were not fluorescent because the fluorescein unit is locked in the closed lactone form. By contrast, compound 202d shows a prominent absorbance band in the visible region (λₘₐₓ=449 nm, ε=27,300±2500 M⁻¹ cm⁻¹) with measurable fluorescence (λₑₘ=514 nm, ϕ=0.11±0.01) due to the free hydroxyl group (Table 1).

TABLE 1
Spectroscopic Properties of compounds 202a-202d
and control compound 204 in PBS (pH 7.4, 10 mM).

| Compound | λₘₐₓ (nm) | ε (M⁻¹cm⁻¹) | λₑₘ (nm) | Φ |
|---|---|---|---|---|
| 202a | n/a | n/a | n/a | n/a |
| 202b | n/a | n/a | n/a | n/a |
| 202c | n/a | n/a | n/a | n/a |
| 202d | 449 | 27,300 ± 2500 | 514 | 0.11 ± 0.01 |
| 204 | n/a | n/a | n/a | n/a |

### Example 7

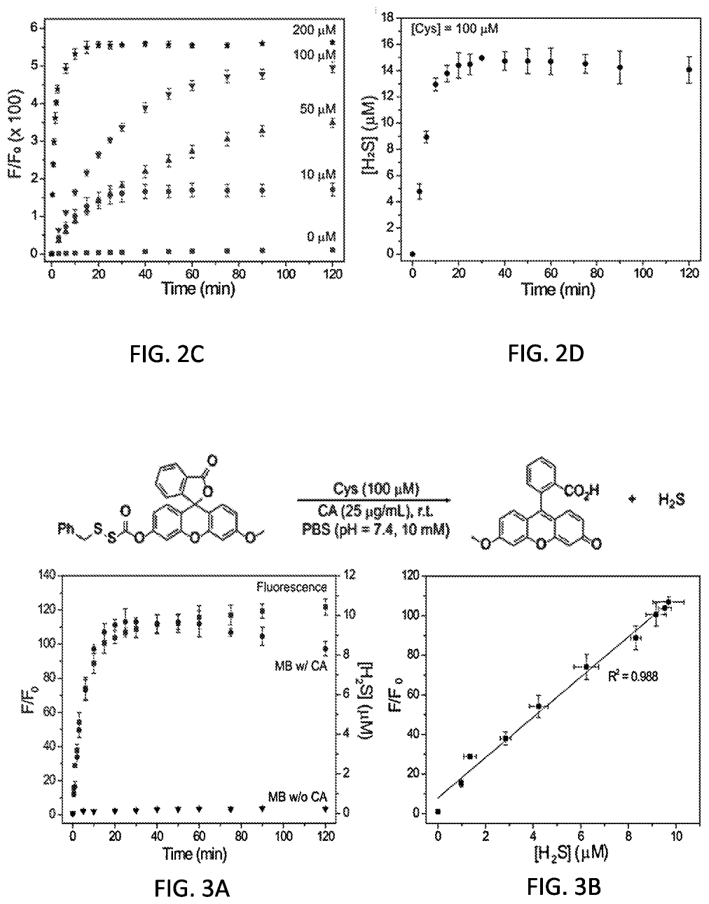

In this example, it was determined that Cys-induced compound 202a activation can be monitored by tracking the fluorescein formation and accompanied fluorescence response, and, in some embodiments, the reaction proceeds more quickly when higher concentrations of thiols are added. Time-dependent fluorescence spectra (FIG. 2A) and UV-vis spectra (FIG. 2B) of a particular compound embodiment, compound 202a, (10 μM) in PBS buffer (pH 7.4, 10 mM) containing Cys (100 μM) and CA (25 μg/mL) were obtained using a fluorescence spectrometer. Cys successfully activated compound 202a and resulted in a 500-fold fluorescence turn on over 2 hours, demonstrating the release of the fluorescein upon compound 202a activation. Fluorescein formation was also confirmed by UV-vis spectroscopy under the identical conditions (FIG. 2B). Specifically, a freshly prepared compound 202a stock solution (3.00 μL, 10.0 mM in DMSO) was added to 3.00 mL of PBS (pH 7.40, 10.0 mM) containing CA (25.0 μg/mL) in a quartz UV cuvette. A Cys stock solution (30.0 μL, 10.0 mM in H₂O) was then added. The absorbance (350-600 nm) was measured for 3 hours using a Cary 100 spectrometer. A Cys-dependent fluorescence enhancement was observed when treating compound 202a (10 μM) with increasing concentrations of Cys (1-20 equiv.), indicating a high sensitivity of compound 202a towards Cys. No fluorescent signal was observed in the absence of Cys, suggesting that compound 202a is stable in aqueous buffer, and that it is not hydrolyzed to provide false signals (FIG. 2C, which shows the Cys-dependent (0-200 μM) fluorescence turn on of compound 202a (10 μM) in PBS). In addition, to confirm the H₂S delivery from compound 202a, compound 202a (10 μM) was treated with Cys (100 μM) in PBS containing CA (25 μg/mL) and quantified H₂S release using the MB assay. 15 μM of H₂S was detected (75% releasing efficiency), which is consistent with our hypothesis that 2 equivalents of COS/H₂S would be released upon compound 202a activation (FIG. 2D, which shows methylene blue (MB) measurement of H₂S release from compound 202a (10 μM) upon Cys (100 μM) activation). These data confirm that COS/H₂S is released from compound 202a upon the Cys activation. Optical measurement conditions were as follows: λₑₘ=490 nm, λₑₓ=500-650 nm, and slit width=0.3 mm.

### Example 8

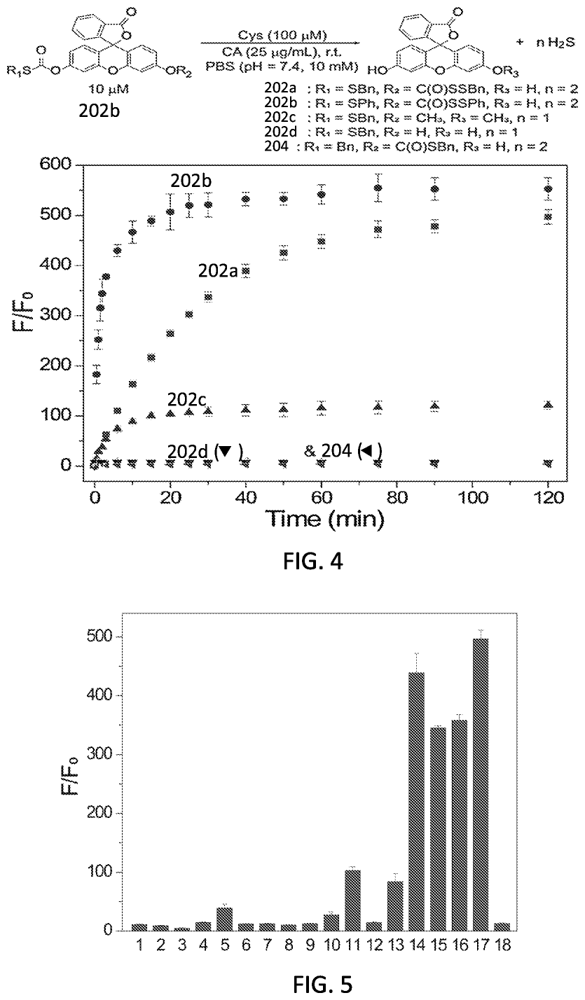

In this example, compound 202c was treated with Cys in PBS containing CA and monitored the fluorescence turn on and H₂S release by fluorescence spectroscopy and MB assay, respectively, to further confirm that both fluorescein and COS/H₂S are generated upon compound activation. To determine whether H₂S release correlated directly with the observed fluorescence response, the fluorescent response from compound 202c in the presence of Cys and CA was measured and quantified H₂S release using the MB assay. For purposes of this example, compound 202c was used because it only contains one sulfenyl thiocarbonate moiety, and therefore should simplify the reaction kinetics. In comparison, compound 202a contains two sulfenyl thiocarbonate groups, and the cleavage of one sulfenyl thiocarbamate would generate compound 202d as a reaction intermediate, which exhibits moderate fluorescence (see FIG. 4, discussed below). Incubation of compound 202c (10 μM) with Cys (100 μM) resulted in a rapid fluorescence response with 96% of the H₂S release measured by MB. At extended time points, a slight decrease in measured H₂S was observed, possibly due to volatilization of H₂S in the headspace of the closed system or adventitious oxidation of released H₂S. Negligible H₂S was detected in the absence of CA, indicating that Cys-triggered H₂S delivery from compound 202c proceeds through intermediate COS formation (FIG. 3A, wherein the trace with the “▪” symbol) shows the fluorescence turn-on results and the trace with the “●” symbol shows the H₂S release detected by MB assay). The fact that no H₂S was detected in the absence of CA indicates that H₂S is released through a COS-dependent pathway (FIG. 3A, trace with “▾” symbol). Optical measurement conditions: λₑₘ=454 nm, λₘₐₓ=500-650 nm, and slit width=0.3 mm. The strong linear correlation between the measured fluorescence and H₂S measured from the MB method detection (first 25 minutes, R²=0.988) demonstrates that fluorescent readouts can serve as reliable optical tools to track COS/H₂S release from compound embodiments with temporal resolution (FIG. 3B, which illustrates the correlation between fluorescence measurement and MB detection). Moreover, this linear correlation suggests that choice of other fluorophores with different brightnesses and photophysical properties could be used to access different dynamic ranges of H₂S release, thus enabling this approach to be translated to different types of experimental designs.

### Example 9

In this example, fluorescence turn on of compound 202a (FIG. 4, indicated with the “▪” symbol), compound 202b (FIG. 4, indicated with the “●” symbol), compound 202c (FIG. 4, indicated with the “▴” symbol) 202d (FIG. 4, indicated with the “▾” symbol), and control compound 204 (FIG. 4, indicated with the symbol “ [symbol] ” in PBS (pH 7.4, 10 mM) containing Cys (100 μM) was evaluated. Treating compounds 202a-202c (10 μM) with Cys (100 μM) in PBS buffer (pH 7.4, 10 mM) resulted in a 120-500-fold fluorescence turn on over 2 hours. Without being limited to a particular theory, it currently is believed that the faster response of compound 202b may be attributed to the more electrophilic phenyl sulfenyl thiocarbonate in comparison to the less electrophilic benzyl sulfenyl thiocarbonate in compound 202a. Compound 202d provided minimal fluorescence enhancement due to its strong background fluorescence. No fluorescence response from compound 204 (10 μM) was observed under the identical conditions, which demonstrates the stability of the thiocarbonate group in the presence of Cys.

### Example 10

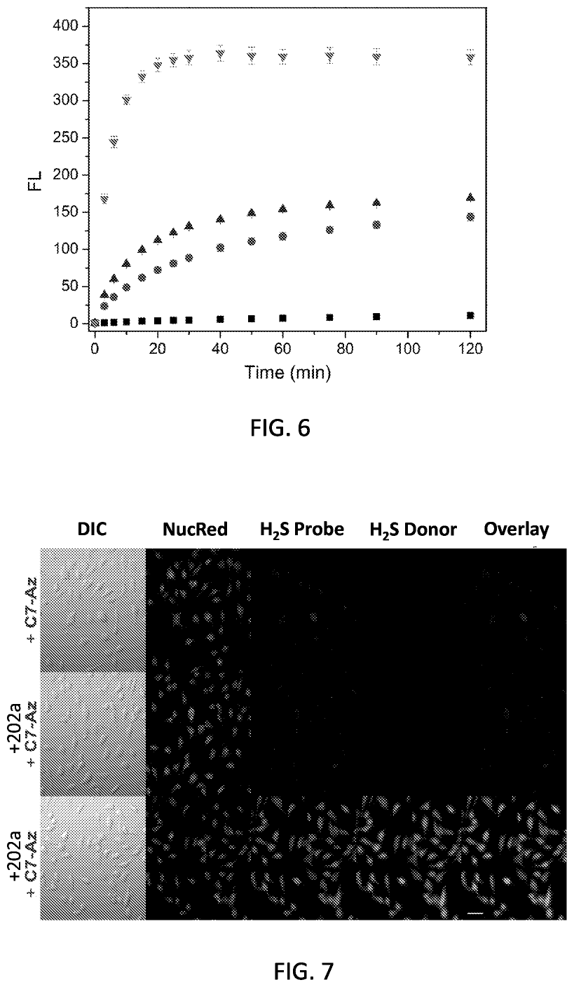

In this example, fluorescence turn on of compound 202a (10 μM) in the presence of cellular reactive sulfur, oxygen, and nitrogen species (RSONs) (100 μM) was evaluated. Results are shown in FIG. 5, wherein bar 1=compound 202a only, bar 2=H₂O₂, bar 3=ClO⁻, bar 4=O₂⁻, bar 5=tert-butyl hydroperoxide (TBHP), bar 6=Ser, bar 7=Lys, bar 8=Gly, bar 9=GSSG, bar 10=GSNO, bar 11=porcine liver esterase (PLE) (1 U/mL), bar 12=bovine serum albumin (BSA), bar 13=penicillamine (PEN), bar 14=N-acetyl cysteine (NAC), bar 15=Hcy, bar 16=GSH, bar 17=Cys, and bar 18=Cys+N-ethylmaleimide (NEM). Compound 202a (10 μM) was incubated with GSH, homocysteine (Hcy), N-acetyl cysteine (NAC), penicillamine (PEN), or bovine serum albumin (BSA) (100 μM), in PBS buffer (pH 7.4, 10 mM) containing CA (25 μg/mL) and the fluorescent intensity was measured after 2 hours. Compound 202a was stable in PBS at physiological pH in the absence of thiols (FIG. 5, bar 1). Incubation of compound 202a with Cys, NAC, GSH, and Hcy, however, led to a significant fluorescence enhancement, indicating successful compound activation and COS/H₂S release (FIG. 5 bars 2-5 and FIG. 6, wherein the “▪” symbol represents results at 0 μM GSH; the “●” represents results at 20 μM GSH; the “▴” symbol represents results at 50 μM GSH; and the “▾” symbol represents results at 100 μM GSH). In addition, these results demonstrate that the sulfenyl thiocarbonate group is responsive to different types of thiols. In comparison, PEN resulted in only minimal fluorescence response and BSA did not activate the compound presumably due to the bulkiness of these two thiol species, which hindered their reactions with compound 202a (FIG. 5, bars 6 and 7). Additionally, N-ethylmaleimide (NEM) pretreatment of Cys samples significantly reduced the fluorescence enhancement from compound 202a, confirming the thiol-induced reduction for the compound activation (FIG. 5, bar 8).

Compound 202a also (10 μM) was incubated with RSONs (100 μM), such as H₂O₂, ClO⁻, O₂⁻, TBHP, Ser, Lys, Gly, GSSG, and GSNO, and COS/H₂S release was monitored by tracking the fluorescein formation. Minimal fluorescence was observed in these embodiments, confirming the stability of compound 202a to common RSONs (FIG. 5, bars 9-17). Because the carbonate functional group may be sensitive to esterase-catalyzed hydrolysis, the esterase stability of the sulfenyl thiocarbonate group also was evaluated by incubating compound 202a (10 μM) with porcine liver esterase (PLE, 1 U/mL). Although a slight fluorescence turn on was observed after a 2-hour incubation, the observed response was much lower than that from thiol activation (FIG. 5, bar 18).

Taken together, the embodiments of this example demonstrate that compound 202a is highly responsive and selective to thiol activation and common cellular RSONs do not trigger compound 202a to release COS/H₂S.

### Example 11

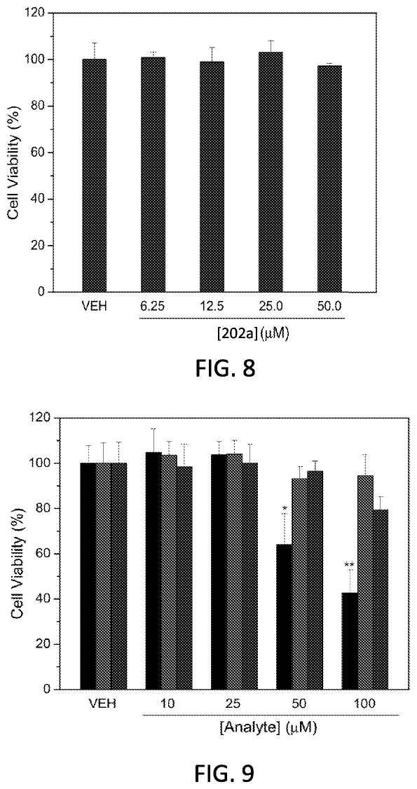

In this example, H₂S delivery from compound 202a in HeLa cells was evaluated. To determine whether compound embodiments of the present disclosure can be activated to release COS/H₂S in cellular environment, HeLa cells were treated with compound 202a under different conditions. HeLa cells were treated with compound 202a (50 μM) or compound 204 (50 μM) and H₂S release was monitored using C7-Az, a H₂S-responsive fluorescent probe. HeLa cells treated with C7-Az (50 μM, an H₂S fluorescent probe) in DMEM did not provide fluorescent signal, indicating that minimum of endogenous H₂S is present in HeLa cells (FIG. 7, top row). HeLa cells treated with the H₂S-responsive fluorescent probe C7-Az (50 μM) and control compound 204 (50 μM) also failed to turn on the fluorescence, suggesting that the thiocarbonate functional group does not provide false positive signals (FIG. 7, middle row). By contrast, a strong C7-Az fluorescent signal was observed when incubating HeLa cells with compound 202a, suggesting that H₂S release was successfully triggered by endogenous thiols. In addition, a strong fluorescence signal was also observed from activated compound 202a in the fluorescein channel, confirming the fluorescence response upon compound activation (FIG. 7, bottom row). To confirm that compound 202a was not cytotoxic, confluent HeLa cells were incubated in FBS-free DMEM containing vehicle (0.5% DMSO), and compound 202a (6.25-50.0 μM) for 30 minutes in a 96-well plate. The culture media were then removed and 100 μL of FBS-free DMEM containing 10% CCK-8 solution was added to each well, and cells were incubated for 2 hours at 37° C. The absorbance at 450 nm was measured by using a microplate reader (see FIG. 8). The cell viability was measured and normalized to the vehicle group. The results are expressed as mean t SEM (n=6).

Taken together, the embodiments of this example demonstrate that the compounds of the present disclosure not only function as efficacious H₂S donors in live cells, but also provide a fluorescence signal that enables observing H₂S release.

### Example 12

In this example, cytoprotective activity of compound 202a against LPS-induced inflammation was evaluated. H₂S has been known to exhibit anti-inflammatory effects by scavenging endogenous nitric oxide (NO). The compounds of the present disclosure were therefore evaluated to determine their anti-inflammatory activities due to H₂S release. RAW 264.7 cells were incubated with compound 202a (0-25 μM) for 2 hours, followed by a 24-hour incubation with lipopolysaccharide (LPS, 0.5 μg/mL) to trigger the inflammatory response. The inflammation event usually results in the NO generation, which can be monitored by measuring nitrite (NO₂—) accumulation. Concentrations of compound 202a of up to 25 μM were evaluated because these concentrations did not induce cytotoxicity. In particular, confluent RAW 264.7 cells were incubated in FBS-free DMEM containing vehicle (0.5% DMSO), compound 202a, BnSH, and compound 204 (10.0-100 μM) for 2 hours in a 96-well plate. The culture media were then removed and 100 μL of FBS-free DMEM containing 10% CCK-8 solution was added to each well, and cells were incubated for 2 hours at 37° C. The absorbance at 450 nm was measured by using a microplate reader (FIG. 9). The cell viability was measured and normalized to the vehicle group. The results are expressed as mean t SEM (n=6).

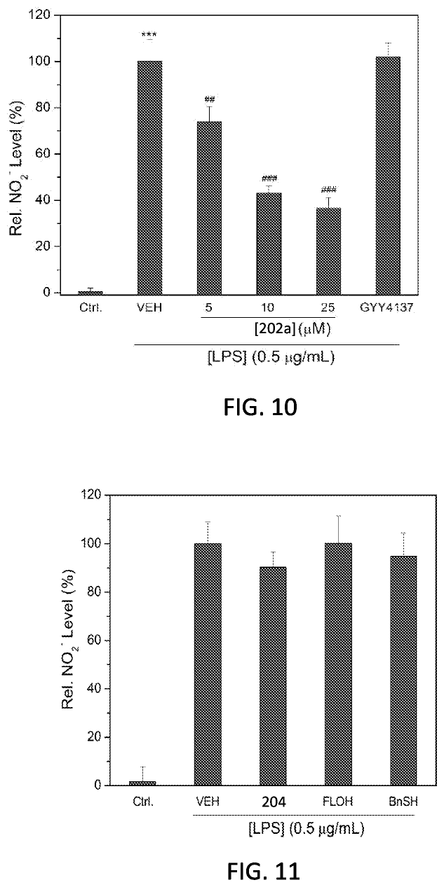

Pretreating RAW 264.7 cells with compound 202a showed a dose-dependent inhibition of NO₂⁻ accumulation, indicating anti-inflammatory activity from compound 202a. Although GYY4137 has shown anti-inflammatory effects at higher concentration and longer incubation time (i.e. 100-1000 μM and 24-hour incubation), such cytoprotection was not observed at the 25 μM concentration used for comparison, highlighting the efficacious H₂S release from compound 202a in the cellular environment (FIG. 10). To further confirm that the observed effects were due to H₂S rather than other components of compound activation, cells were treated with 25 μM of compound 204, fluorescein or benzyl mercaptan and NO₂⁻ production was measured. In particular, Macrophage RAW 264.7 cells were seeded in a 24-well plate (5×105 cells/well) containing 0.500 mL of DMEM and incubated at 37° C. under 5% CO₂ for 24 h. The confluent cells were washed with PBS and incubated with 25 μM of compound 204, fluorescein (FLOH), or benzyl mercaptan (BnSH) at 37° C. for 2 hours. Compounds were then removed by washing cells with PBS and these pretreated cells were incubated in FBS-free DMEM containing LPS (0.500 μg/mL) for 24 hours. NO₂⁻ levels were measured by using a Griess Reagent Kit. As expected, none of these species exhibited anti-inflammatory activities.

None of these compounds attenuated NO₂⁻ generation, confirming that the anti-inflammatory activities of compound 202a is due to H₂S release (FIG. 11).

Overall, the embodiments of this example demonstrate that compound 202a releases COS/H₂S in complex cellular environment and exhibits promising anti-inflammatory protections, indicating potential applications of compound 202a as H₂S-releasing therapeutics.

### Example 13

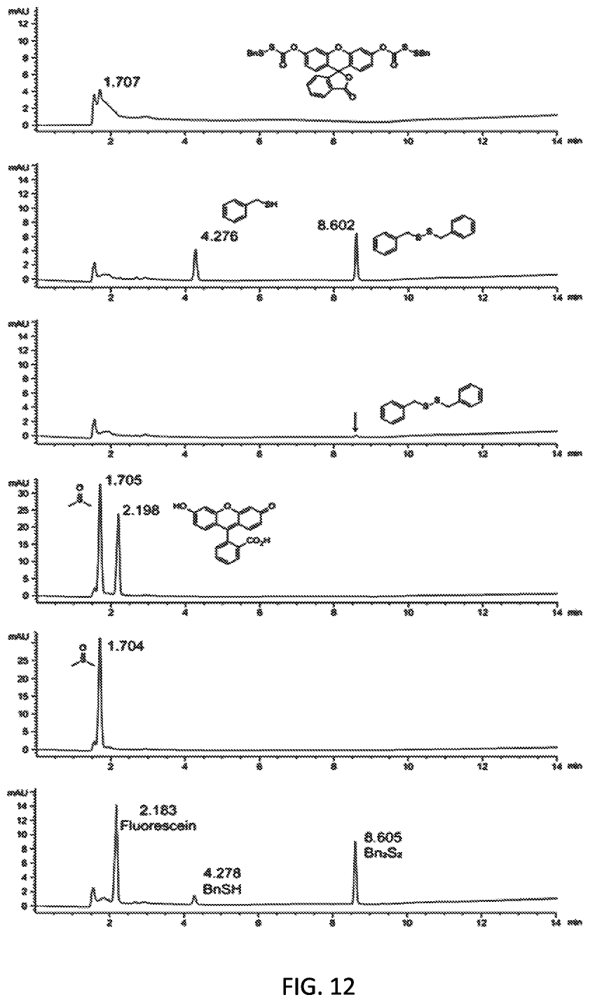

In this example, compound 202a (10 μM) was incubated in PBS (pH 7.4, 10 mM) with 10 equivalents of benzyl mercaptan (100 μM) for 1 hour and the reaction products were analyzed by HPLC. To a 3.00 mL PBS (pH 7.4, 10 mM) containing benzyl mercaptan (100 μM), 3.00 μL of 202a (10 mM in THF) was added and stirred at room temperature. After 1 hour, a 1 mL reaction aliquot was analyzed by HPLC. HPLC analysis was performed on an Agilent 1260 HPLC instrument with a Poroshell 120 EC-C18 4.6×100 mm column and monitored absorption at 230 nm. HPLC Method: Solvent A: 95% H₂O, 5% MeOH, Solvent D: 100% MSCN. Gradient: 35% Solvent A/65% Solvent B for 2 minutes. Change to 100% Solvent B over 4 minutes and hold for 6.5 minutes. Change to 35% Solvent A/65% Solvent B over 0.5 minutes and hold for 4.5 minutes. Flow Rate: 0.5 mL/min, 2 μL injection, unless stated otherwise. To confirm the formation of expected reaction products and confirm observed peaks, authentic samples of 20 μM benzyl disulfide, 10 μM compound 202a, and 100 μM benzyl mercaptan were prepared in 10 mM PBS (pH 7.4) containing 0.1-1.0% THF and analyzed as described above. Due to the low absorption of compound 202a at 230 nm, injection volume was increased to 8.0 μL. Due to poor solubility in THF, an authentic sample of 10 μM fluorescein was prepared in 10 mM PBS (pH 7.4) containing 0.1% DMSO. The results are shown in FIG. 12. As shown by FIG. 12, compound 202a consumption and the formation of both benzyl disulfide and fluorescein was observed.

### Example 14

In this example, GSH-dependent H₂S release from compound 306a was evaluated. Compound 306a was synthesized by reacting thiobenzamide with chlorocarbonylsulfenyl chloride reagent. Briefly, thiobenzamide was dissolved in THF, followed by the addition of sulfenyl chloride reagent. The resultant solution was stirred at room temperature for 2 hours. The solvent was removed under vacuum and the product was isolated as yellow solid by column chromatography.

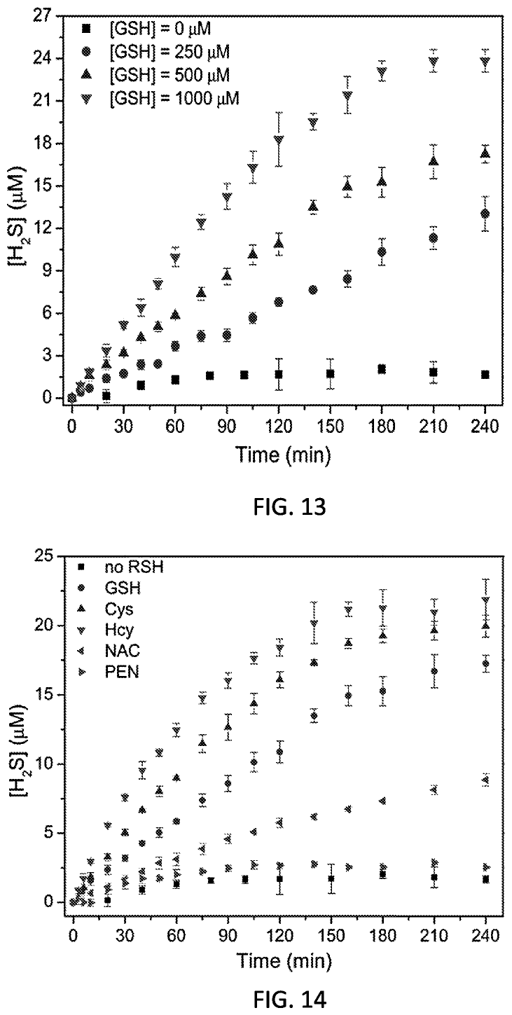

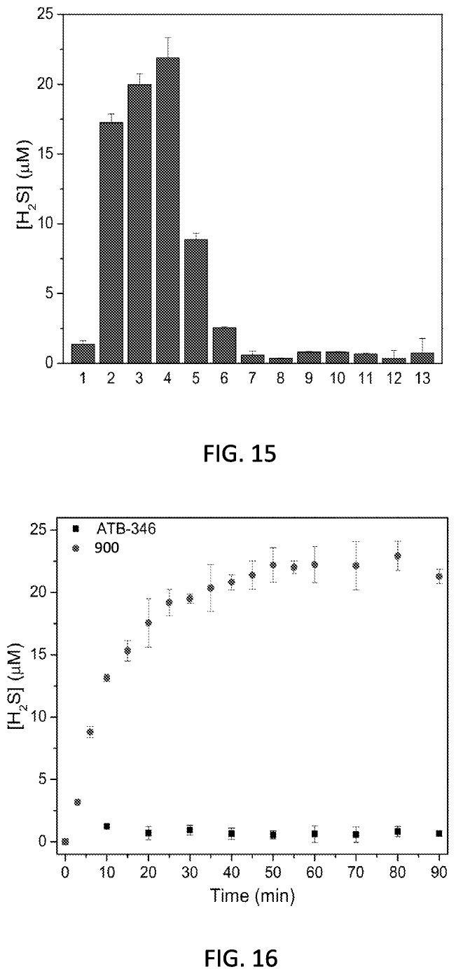

To evaluate thiol effects on COS/H₂S release from cyclic perthiocarbamate compound embodiments, compound 306a (50 μM) was incubated in PBS (pH 7.4, 10 mM) containing Cys or GSH (up to 4.0 mM) and CA (25 μg/mL) and H₂S release was monitored by MB assay. A GSH-dependent H₂S release was observed, indicating a successful GSH-induced compound activation. Results are shown in FIG. 13. Additionally, other thiol triggers, such as cysteine, homocysteine, and N-acetyl cysteine, were combined with compound 306a to evaluate if they would trigger the compound to release H₂S. Negligible H₂S release was observed in the absence of thiol trigger, indicating the compound did not release H₂S by hydrolysis. Penicillamine resulted in minimal H₂S release due to the bulkiness of the thiol trigger. Results are shown in FIG. 14. The compound did not release H₂S spontaneously in aqueous buffer. In the presence of thiols, such as GSH, Cys, Hcy, and NAC, H₂S release was observed. As shown in FIG. 15, PEN triggered a much less efficient H₂S release due to the bulkiness of the thiol trigger (bars 1-6). Other cellular labile species, such as GSSG, Lys, Ser, Gly, S₂O₃²⁻, SO₃²⁻, and SO₄²⁻, did not activate the compound and minimal H₂S release was observed (bars 7-13).

### Example 15

In this example, GSH-triggered H₂S release from compound 900 was evaluated. ATB-346 has been demonstrated to exhibit promising anti-inflammatory effect with much reduced side effects in the GI system, presumably due to H₂S release, although the H₂S release from thioamide is inefficient. To improve the H₂S releasing capacity of ATB-346, compound 900 was made by reacting ATB-346 with chlorocarbonyl sulfenyl chloride reagent (see Scheme 9). The resultant compound 900 was then added to PBS containing GSH and CA. A promising H₂S release was observed (see FIG. 16), which was not detected from ATB-346 under the identical conditions. This experiment indicates that compound 900 is a potent H₂S donor and may have potential anti-inflammatory effects.

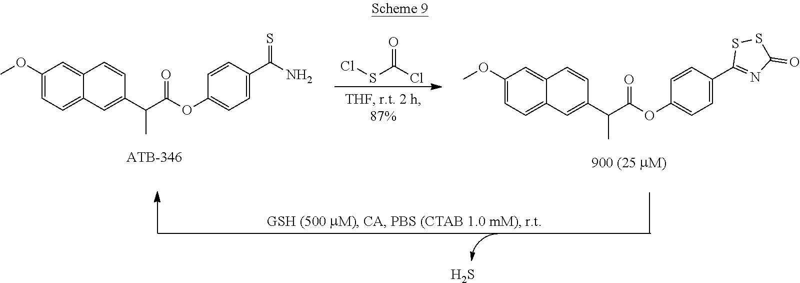

### Example 16

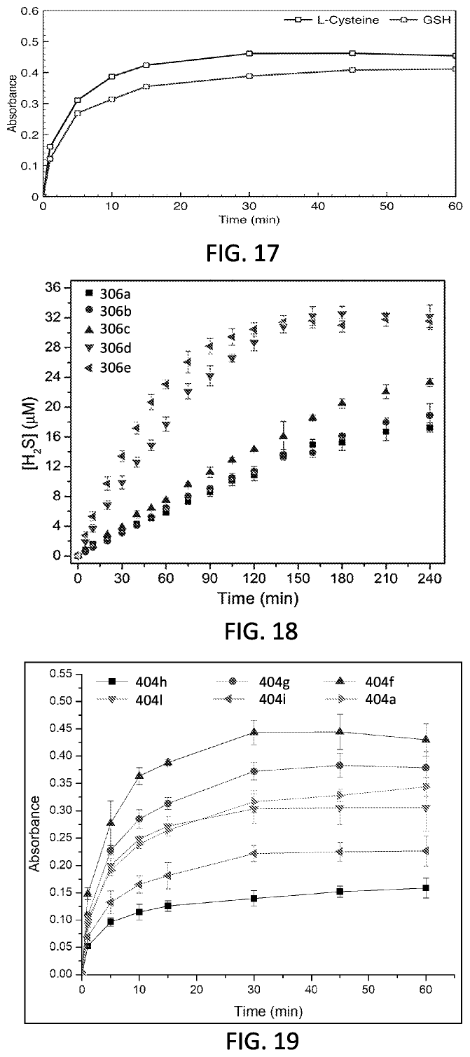

In this example, it was shown that compound 404c reacts with thiols to release two equivalents of COS, which is converted to H₂S by the ubiquitous enzyme carbonic anhydrase (CA). Release of H₂S from compound 404c (25 μM) in the presence of L-cysteine or reduced glutathione (GSH) (500 μM, 20 equiv.) measured by the spectrophotometric methylene blue assay, with results shown in FIG. 17.

### Example 17

In this example, additional compounds, such as cyclic sulfenyl thiocarbamate compounds 306a-306e, were evaluated for their ability to release H₂S upon exposure to a biological thiol, cysteine. All of these compound embodiments exhibited good H₂S release, as can be seen by FIG. 18. Results for additional compound embodiments also are shown in FIG. 19.

### Example 18

In this example, H₂S delivery from sulfenyl thiocarbamate compound 306a in HeLa cells was evaluated. HeLa cells were treated with Hoechst dye and SF7-AM (5 μM) in DMEM only for 5 minutes and then with DMEM only for 30 minutes (top row) or DMEM containing the compound (50 μM) for 30 minutes (bottom row). Cells were then washed with PBS and cell images were taken in PBS using a fluorescent microscope.

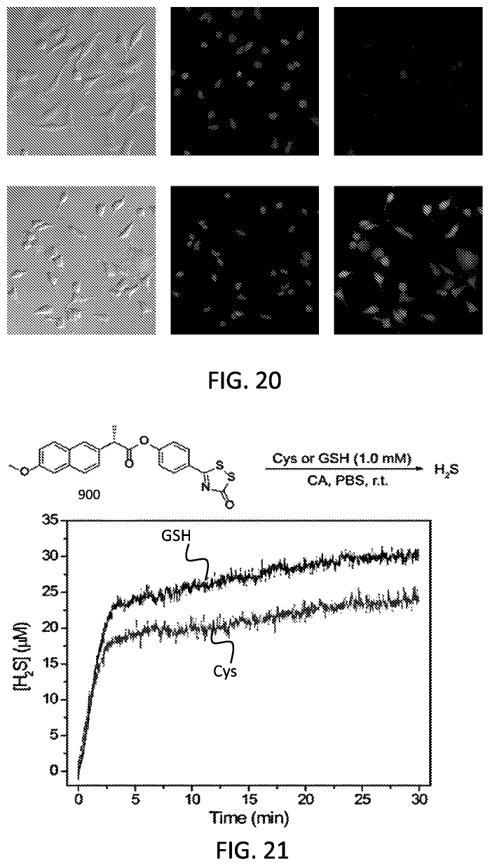

Incubation of HeLa cells with SF-7AM (5 μM), an H₂S fluorescent probe, resulted in a negligible fluorescence response, indicating minimal endogenous H₂S (FIG. 20, top row). By contrast, a strong SF7-AM fluorescent signal was observed when incubating HeLa cells with the H₂S donor, suggesting that H₂S release was successfully triggered by endogenous thiols (FIG. 20, bottom row).

### Example 19

H₂S Hybrid non-steroidal anti-inflammation drugs (H₂S-NSAIDs) have been developed by coupling H₂S donors and regular NSAIDs in recent years and the resultant H₂S-NSAIDs, compared to parent NSAIDs, have been demonstrated to exhibit potent anti-inflammation activities with reduced side damages in the GI system due to H₂S release although H₂S release from these compound motifs remains in debate.

In this example, H₂S-NSAID was synthesized by using naproxen as the model NSAID (see Scheme 6). Briefly, naproxen, HOBt, EDC, and DMAP were added to CH₃CN and the solution was stirred at room temperature for 30 minutes, followed by the addition of 4-hydroxythiobenzamide. The reaction solution was stirred at room temperature for 18 hours. The solvent was removed and the product, ATB-346, was isolated as yellow solid (42%). ATB-346 was then added to THF, followed by the addition of chlorocarbonyl sulfenyl chloride. The solution was stirred at room temperature for 2 hours and the solvent was then removed under vacuum. The final product, 900, was isolated by column chromatography (87%).

With the hybrid NSAID in hand, COS/H₂S release in aqueous buffer was evaluated. 900 (50 μM) was incubated in PBS buffer (pH 7.4, 10 mM) containing CTAB (1.0 mM) and CA (25 μg/mL) and the thiol-triggered H₂S release was immediately observed when Cys or GSH (1.0 mM) was added (FIG. 21).

### Example 20

In this example, a compound is administered to a subject by preparing a pharmaceutical composition comprising the compound and a pharmaceutically acceptable excipient. The composition is administered either by administering an oral dosage form comprising the composition to the subject, by injecting the composition at a site of interest, by intraperitoneal injection, or by applying a topical ointment comprising the composition at a site of interest. The subject is evaluated for an increase in concentration of H₂S by taking a blood sample from the subject and determining the concentration of H₂S in the blood sample as compared to a blood sample taken from the subject prior to administration of the pharmaceutical composition comprising the compound.

### Example 21

In this example, a compound is administered to a sample by exposing the sample to a composition comprising the compound. The sample is then optionally exposed to a separate composition comprising a reactive compound. The sample is evaluated to determine if a detectable signal is emitted within the sample after exposure to the composition comprising the compound. In some embodiments, a fluorescence assay is used. The evaluation step can comprise analyzing the sample using a spectrofluorometer, a fluorescent microscope, a fluorescence scanner, or a flow cytometer.

In view of the many possible embodiments to which the principles of the present disclosure may be applied, it should be recognized that the illustrated embodiments are only preferred examples and should not be taken as limiting in scope. Rather, the scope of the present disclosure is defined by the following claims. We therefore claim as our invention all that comes within the scope and spirit of these claims.

1. A compound having a structure satisfying Formula I 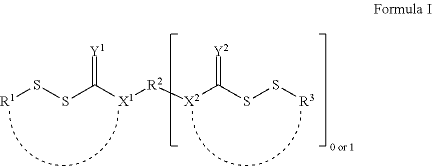
wherein:
R¹ and R³ independently are an organic functional group, aromatic, aliphatic, heteroaliphatic, haloaliphatic, haloheteroaliphatic, or any combination thereof; or R¹ and X¹, together, provide a 5-membered ring and/or R³ and X², together, provide a 5-membered ring;
X¹ and X² independently are oxygen, nitrogen, or NR⁵, wherein R⁵ is hydrogen, aliphatic, heteroaliphatic, haloaliphatic, haloheteroaliphatic, aromatic, an organic functional group, or a combination thereof;
each of Y¹ and Y² independently is oxygen or sulfur; and
R², if present, is aromatic, aliphatic, heteroaliphatic, haloaliphatic, haloheteroaliphatic, hyrogen, an organic functional group, or a combination thereof.

2. The compound of claim 1, wherein the compound has a structure satisfying Formula V 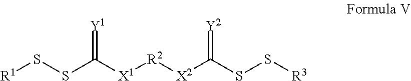
wherein R¹ and R³ independently are aromatic or an organic functional group; X¹ and X² independently are oxygen or NR⁵; and R² is aromatic or heteroaliphatic.

3. The compound of claim 2, wherein R¹ and R³ independently are benzyl or phenyl and R² is heteroaryl or heterocyclic.

4. The compound of claim 2, wherein X¹ and X² are oxygen and R² is

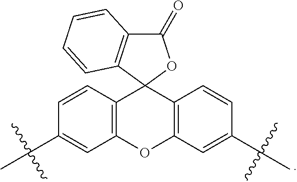 [SMILES: *c1ccc2c(c1)Oc1cc(*)ccc1C21OC(=O)c2ccccc21]

5. The compound of claim 1, wherein R¹ is C(O) and X¹ is N, and wherein R¹ and X¹, together, provide a 5-membered ring wherein R¹ is bound to X¹ via a single bond, thereby providing a compound having a structure satisfying Formula III 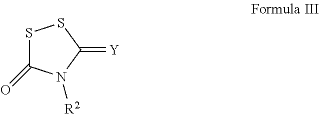
wherein R² is aliphatic, heteroaliphatic, haloaliphatic, haloheteroaliphatic, aromatic, an organic functional group, or a combination thereof; and Y is oxygen or sulfur.

6. The compound of claim 5, wherein R² is heteroaryl-(Z)ₙ or -aryl-(Z)ₙ, wherein each Z is a substituent other than hydrogen and n is an integer ranging from 0 to 20; alkyl; or a heterocyclic group.

7. (canceled)

8. The compound of claim 6, wherein each Z independently is aliphatic; aromatic; heteroaliphatic selected from peroxy, disulfide, alkoxy, ether, thioether, or amino; haloaliphatic; haloheteroaliphatic; an organic functional group selected from aroxy, aldehyde, acyl halide, halogen, nitro, cyano, azide, carboxyl (or carboxylate), amide, ketone, carbonate, imine, azo, carbamate, hydroxyl, thiol, sulfonyl (or sulfonate), oxime, ester, thiocyanate, thioketone, thiocarboxylic acid, thioester, dithiocarboxylic acid or ester, phosphonate, phosphate, silyl ether, sulfinyl, thial, or combinations thereof; or any combination thereof.

9. (canceled)

10. The compound of claim 1, wherein the compound has a structure satisfying Formula IV 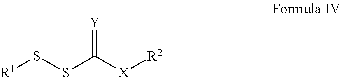
wherein R¹ is aliphatic, heteroaliphatic, haloaliphatic, haloheteroaliphatic, aromatic, an organic functional group, or any combination thereof; and R² is hydrogen, aliphatic, heteroaliphatic, haloaliphatic, haloheteroaliphatic, aromatic, an organic functional group, or a combination thereof; and Y is oxygen or sulfer.

11. The compound of claim 10, wherein R¹ is phenyl or benzyl; X is oxygen; and wherein R² is

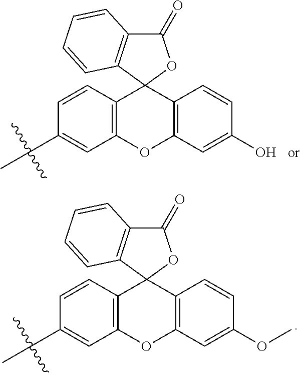 [SMILES: *c1ccc2c(c1)Oc1cc(O)ccc1C21OC(=O)c2ccccc21 ; *c1ccc2c(c1)Oc1cc(OC)ccc1C21OC(=O)c2ccccc21]

12. The compound of claim 11, wherein R¹ is 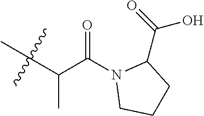 [SMILES: *C(C)C(=O)N1CCCC1C(=O)O]
and, X is oxygen, and R² is alkyl.

13. The compound of claim 1, wherein R¹ is CR⁴ and X¹ is nitrogen and R¹ and X¹, together, provide a 5-membered ring wherein R¹ is bound to X¹ via a double bond, thereby providing a compound having a structure satisfying Formula II 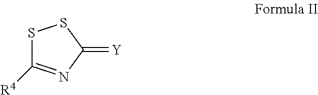
wherein R⁴ is aliphatic, heteroaliphatic, haloaliphatic, haloheteroaliphatic, aromatic, an active agent, an organic functional group, or any combination thereof; and Y is oxygen or sulfur.

14. The compound of claim 13, wherein R⁴ is -heteroaryl-(Z)ₙ or -aryl-(Z)ₙ, wherein each Z independently is selected from aliphatic; aromatic; heteroaliphatic selected from peroxy, disulfide, alkoxy, ether, thioether, or amino; haloaliphatic; haloheteroaliphatic; an active agent; an organic functional group selected from aroxy, aldehyde, acyl halide, halooen, nitro, cyano, azide, carboxyl (or carboxylate), amide, ketone, carbonate, imine, azo, carbamate, hydroxyl, thiol, sulfonyl (or sulfonate), oxime, ester, thiocvanate, thioketone, thiocarboxylic acid, thioester, dithiocarboxylic acid or ester, phosphonate, phosohate, silyl ether, sulfinyl, thial, or combinations thereof; or any combination thereof; and n is an integer ranging from 0 to 20.

15-16. (canceled)

17. A compound having a structure satisfying any one or more of Formulas IIA-VA, 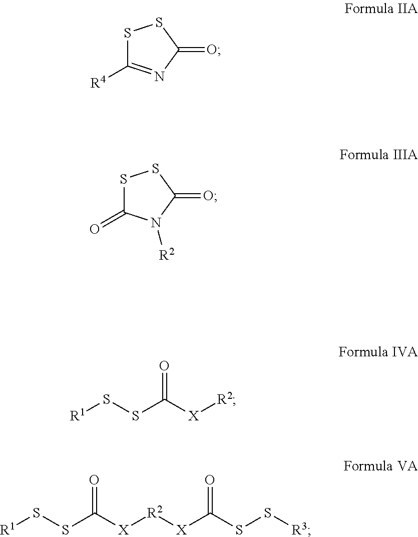
wherein:
R¹ and R³ independently are aliphatic, heteroaliphatic, haloaliphatic, haloheteroaliphatic, aromatic, or any combination thereof; or R¹ (and/or R³) can join with an X group to provide a ring;
each X independently is oxygen or NR⁵, wherein R⁵ is hydrogen, aliphatic, heteroaliphatic, aromatic, or any combination thereof;
R² is aromatic aliphatic, heteroaliphatic, haloaliphatic, haloheteroaliphatic, hydrogen, or any combination thereof when the compound has a structure satisfying Formula IV; or R² is aliphatic, heteroaliphatic, haloaliphatic, haloheteroaliphatic, aromatic, or any combination thereof when the compound has a structure satisfying Formulas III or V; and
R⁴ is aliphatic, heteroaliphatic, aromatic, or any combination thereof.

18. The compound of claim 1, wherein the compound is

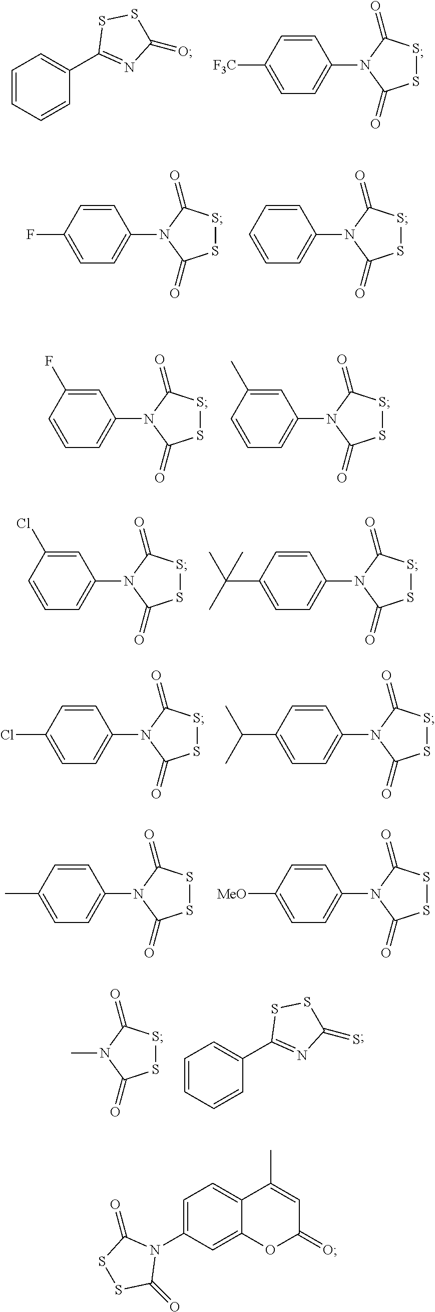

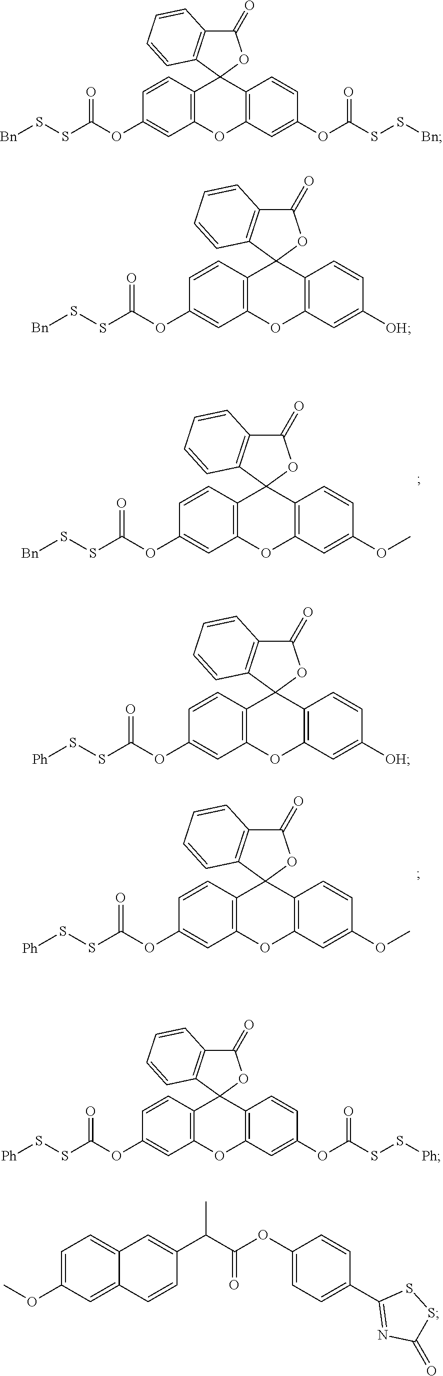

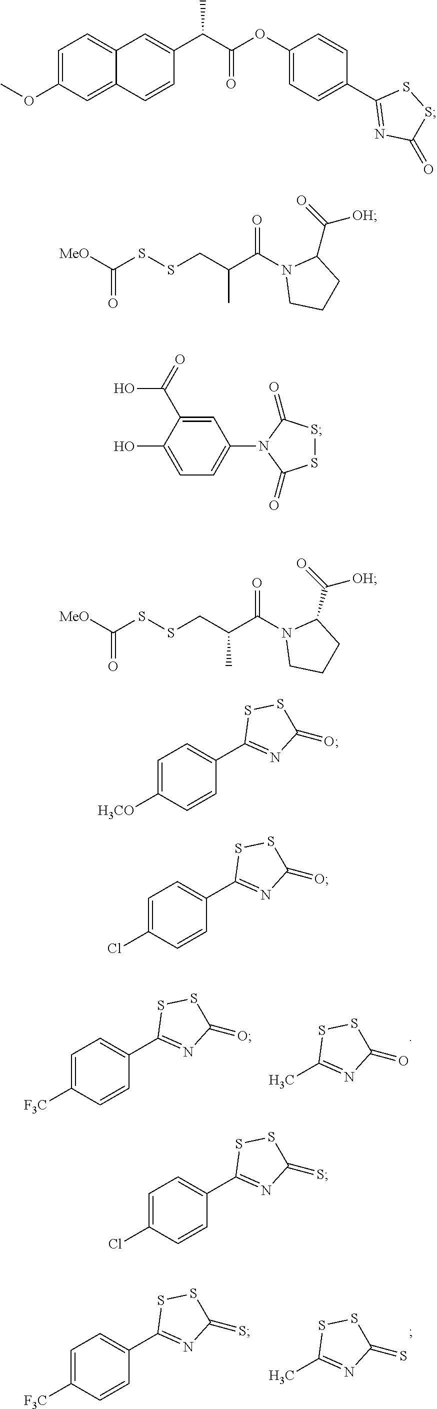

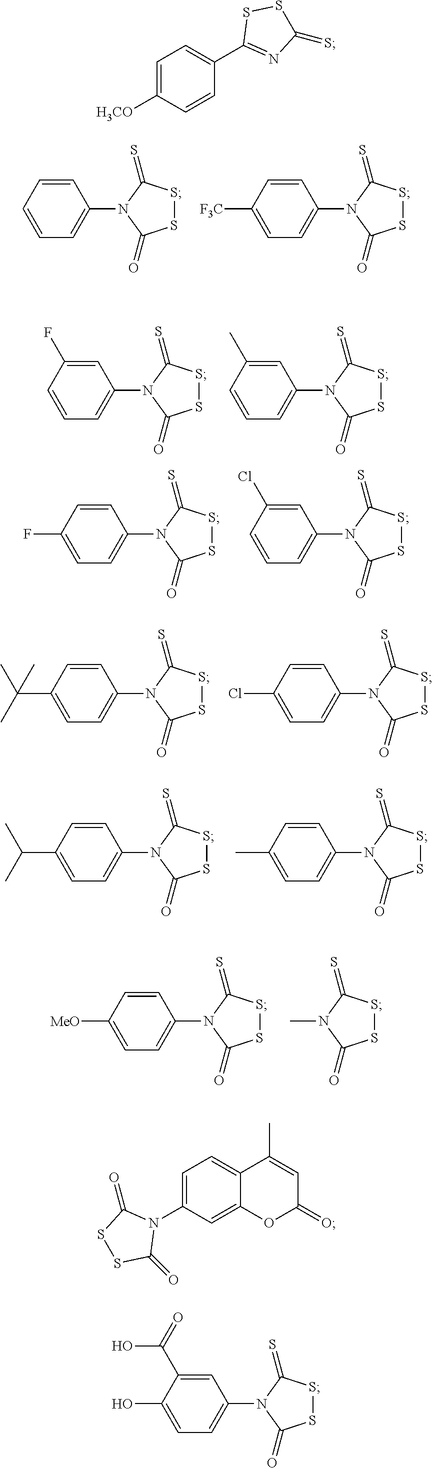

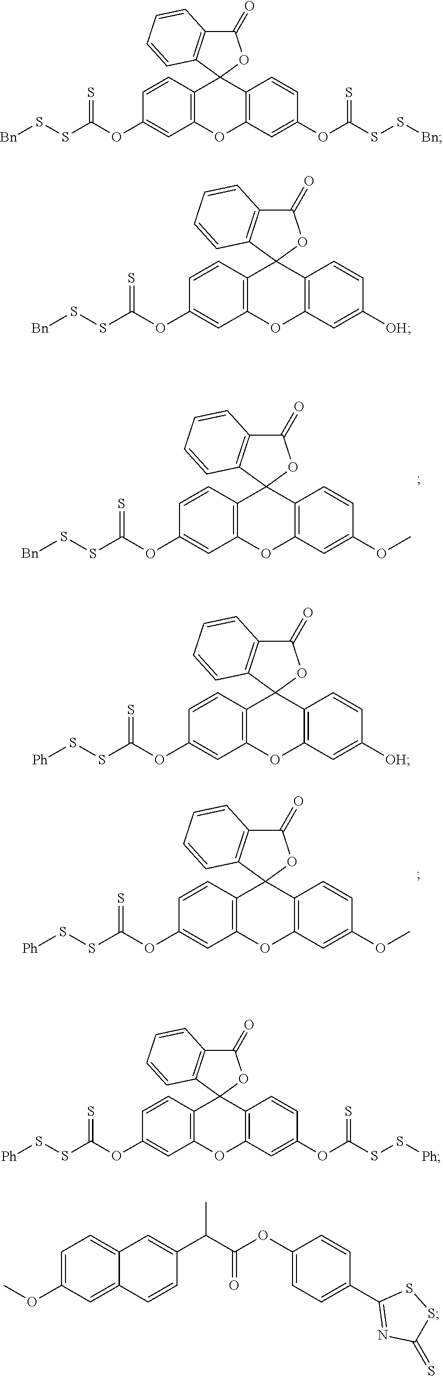

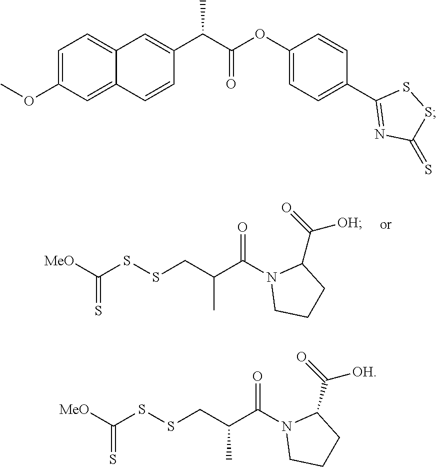

19. A pharmaceutical composition, comprising a compound of claim 1, and a pharmaceutically acceptable excipient.

20. A method, comprising exposing a sample or a subject to a compound according to claim 1, or a pharmaceutical composition thereof.

21. The method of claim 20, further comprising analyzing the sample or the subject to detect a reaction between the compound and a thiol-containing compound that is inherently present in the subject or the sample or that is added to the subject or the sample, wherein the reaction produces a detectable signal, COS, H₂S, or a combination thereof.

22. The method of claim 21, wherein analyzing comprises detecting and/or measuring a fluorescence change, or a change in concentration of H₂S, or any combination thereof.

23. The method of claim 20, further comprising measuring an amount of H₂S released from the compound.

24. The method of claim 20, wherein the sample is a biological sample selected from a cell, tissue, and/or bodily fluid.

25. The method of claim 20, wherein the method comprises exposing a subject that has or is at risk of developing a disease associated with H₂S deficiency or H₂S misregulation.

26. The method of claim 25, wherein the disease is diabetes, inflammation, a neurological disease, cancer, a disease involving insufficient wound healing, erectile dysfunction, a cardiovascular disease selected from heart failure, myocardial reperfusion injury, atherosclerosis, hypertension, hypertrophy, or any combinations thereof; or any combination of such diseases.

27. (canceled)
